(19) United States
(12) Patent Application Publication
(10) Pub. No.: US 2007/0292917 A1
(43) Pub. Date: Dec. 20, 2007

(54) COMPOSITION AND METHODS FOR EXPRESSING REPORTER MOLECULES IN MAMMALIAN CELLS

(75) Inventors: George Hanson, Madison, WI (US); David Thompson, Monona, WI (US)

Correspondence Address: INVITROGEN CORPORATION;C/O INTELLEVATE, P.O. BOX 52050, MINNEAPOLIS, MN, 55402, US

(73) Assignee: Invitrogen Corporation, Carlsbad, CA (US)

(21) Appl. No.: 11/766,003

(22) Filed: Jun. 20, 2007

Related U.S. Application Data
(60) Provisional application No. 60/805,297, filed on Jun. 20, 2006.
(60) Provisional application No. 60/889,913, filed on Feb. 14, 2007.

(51) Int. Cl. C12P 21/06; C12N 15/86; C12N 7/00

(52) U.S. Cl. 435/69.1; 435/456; 435/235.1

(57) ABSTRACT

Disclosed herein is a novel system and methods for expressing exogenous genes, such as genes encoding fluorescent proteins, in mammalian cells. In one embodiment of this system and methods, a gene essential for viral infectivity or replication in cell culture is deleted or inactivated in the genome of a non-mammalian DNA virus. The exogenous gene operably linked to a mammalian promoter is then inserted into the non-replicative non-mammalian DNA virus. The non-replicative virus is propagated in a host cell that expresses in trans the deleted or inactivated gene or a functional homolog.

### RELATED APPLICATIONS

This application claims the benefit under 35 U.S.C. 119(e) of U.S. Provisional Patent Application Nos. 60/805,297, filed Jun. 20, 2006, and 60/889,913, filed Feb. 14, 2007, the entire disclosures of which are incorporated herein by reference.

### FIELD OF THE INVENTION

The invention pertains to the use of viruses to express exogenous genes in cells that are not natural hosts for the viruses.

### BACKGROUND OF THE INVENTION

Important breakthroughs in medicine and medical diagnostics are facilitated by an increased understanding of biological processes that occur within cells. To better understand these processes, scientists utilize reporter molecules. Some reporter molecules can be used to make biosensors, which can be used to monitor the presence, concentration changes, and movement of various biochemicals within a cell.

Geneticaly encoded reporter molecules, such as genetically encoded fluorescent proteins (FPs), are commonly used by scientists to study cell function. However, in order to facilitate the development and commercial availability of such genetically encoded reporters, sufficient financial incentive must exist for companies that sell such reagents. The challenge of commercializing genetically encoded reporters such as FPs is that supplying the FPs via a DNA vector allows end users to very easily transform bacteria and produce more of the vector, in effect resulting in a one time sale of the reporter molecule. The one-time sale nature of DNA vectors encoding reporter molecules makes it difficult to financially justify the considerable time and expense in research and development to develop such a vector. Moreover in the case of FPs the end-user often needs to deliver the FP encoding vector to mammalian cells, which can often prove to be a non-trivial task.

Current methods for expressing an exogenous gene in a mammalian cell include the use of mammalian viral vectors, such as those which are derived from retroviruses, adenoviruses, herpes viruses, vaccinia viruses, polio viruses, or adeno-associated viruses. Other methods of expressing an exogenous gene in a mammalian cell include direct injection of DNA, the use of ligand-DNA conjugates, the use of adenovirus-ligand-DNA conjugates, calcium phosphate precipitation, and methods which utilize a liposome- or polycation-DNA complex. In some cases, the liposome- or polycation-DNA complex is able to target the exogenous gene to a specific type of tissue, such as liver tissue. Some methods of targeting genes to liver cells utilize the asialoglycoprotein receptor (ASGP-R) which is present on the surface of hepatocytes (Spiess et al., 1990, Biochem. 29:10009-10018). The ASGP-R is a lectin which has affinity for the terminal galactose residues of glycoproteins. In these cases, the DNA complexes are endocytosed by the cell after they are bound to the ASGP-R on the cell surface.

Non-mammalian viruses have been used to express exogenous genes in non-mammalian cells. For example, viruses of the family Baculoviridae (commonly referred to as baculoviruses) have been used to express exogenous genes in insect cells. One of the most studied baculoviruses is the Autographa californica multiple nuclear polyhedrosis virus (AcMNPV). Although some species of baculoviruses which infect crustacea have been described (Blissard, et al., 1990, Ann. Rev. Entomology 35:127), the normal host range of the baculovirus AcMNPV is limited to the order lepidoptera.

Baculoviruses are large double stranded DNA viruses that are pathogens of insects. Infection of the host begins when insect larvae acquire the virus orally. Infection is first observed in the epithelial cells of the midgut and is followed in most cases by systemic infection. One hallmark of the baculovirus infection cycle is the production of two structurally and functionally distinct virion phenotypes. One virion phenotype, the occlusion derived virus (ODV), is found within the protective occlusion bodies. Once released from the occlusion body by the alkaline pH of the gut, the ODV initiates infection of the animal by infecting epithelial cells of the midgut. A second virion phenotype, the budded virus (BV), is produced by budding from the surface of infected cells. The BV is initially produced from infected midgut epithelial cells and is essential for systemic infection, mediating movement of the virus from midgut to other tissues and propagating the infection from cell to cell within the infected animal. BV are highly infectious to tissues of the hemocoel and to cultured cells, whereas ODV appear to be less infectious in cell culture or when injected into the hemocoel. The two virion phenotypes also differ in entry mechanisms, as the BV enter cells via endocytosis, while the ODV appear to fuse directly with the plasma membrane at the cell surface.

The major envelope protein of the BV is the GP64 Envelope Fusion Protein (GP64 EFP, also known as GP64 or GP67), which is an extensively processed type I integral membrane glycoprotein that has been studied in some detail. Densely packed peplomers found on the surface of BV are believed to be composed of the GP64 EFP protein and these peplomers are acquired by the virion during budding. Recent studies of a soluble form of GP64 EFP indicate that the native form of GP64 EFP is trimeric and thus, each peplomer is likely comprised of a single trimer of GP64 EFP. The important role of GP64 EFP in BV infectivity is demonstrated by the neutralization of BV infectivity with antibodies specific to GP64 EFP. Using syncytium formation assays and cells expressing gp64 EFP, it was shown that the GP64 EFP protein is both necessary and sufficient for low pH activated membrane fusion activity. In addition, two functional domains have been identified in GP64 EFP: an oligomerization domain necessary for trimerization and transport, and a small internal hydrophobic membrane fusion domain. Thus, functional studies of GP64 EFP show that GP64 EFP mediates membrane fusion in a pH dependent manner, consistent with an essential role for GP64 EFP during viral entry by endocytosis.

### SUMMARY OF THE INVENTION

The invention is based in part on the discovery that second messenger system proteins and fluorescent proteins, especially fluorescent proteins that make up at least a portion of a cell sensor, can be advantageously provided as research tools by encoding them within recombinant viruses that naturally infect a natural host species, wherein an end user uses the reconbiment virus to infect cells other than the natural host species. For example, the invention provides non-mammalian DNA viruses for use as tools to infect mammalian cells and deliver a second messenger system protein, fluorescent protein, or genetically-encoded sensor to the mammalian cells. An advantage in this configuration as a research tool is that it is not convenient for an end user to replicate the virus, thus making it more convenient for the end user to purchase more recombinant virus stock from a provider, thus making the product more like a more profitable consumable-type product, rather than a replicable product. Furthermore, the virus can be rendered even less-easily replicable by an end user by using a mutant virus wherein a gene required for replication is deleted or inactivated.

In illustrative embodiments, the present invention provides a non-replicative form of a virus such as a baculovirus that has a genetically encoded reporter integrated into its genome, that is operably linked to a mammalian promoter. By “promoter” is meant at least a minimal sequence sufficient to direct transcription. A “mammalian-active” promoter is one that is capable of directing transcription in a mammalian cell. The term “mammalian-active” promoter includes promoters that are derived from the genome of a mammal, i.e., “mammalian promoters,” and promoters of viruses that are naturally capable of directing transcription in mammals (e.g., an MMTV promoter, a CMV promoter or a hepatitis viral promoter). The non-replicative virus is used to provide a means to easily deliver and express the reporter in a mammalian cell in a commercially attractive manner. Accordingly, one embodiment of the invention is a composition comprising a genome of a non-mammalian DNA virus wherein a gene required for replication in a non-mammalian host cell is deleted or inactivated and further comprising an exogenous gene operably linked to a mammalian promoter.

Another embodiment of the invention is a method for expressing an exogenous gene in a mammalian cell comprising inserting the exogenous gene into the genome of a non-mammalian DNA virus wherein the exogenous gene is operably linked to a mammalian promoter and wherein a gene required for replication in a non-mammalian host cell is deleted or inactivated; then harvesting the non-mammalian DNA virus and contacting the non-mammalian DNA virus with the mammalian cell such that the non-mammalian DNA virus infects the mammalian cell and the exogenous gene is expressed. In illustrative embodiments, the exogenous gene is a reporter gene, such as a gene encoding a fluorescent protein or a gene encoding a fluorescent sensor molecule, such as a fluorescent sensor molecule comprising a fluorescent protein.

In some embodiments, the method further comprises propagating the non-mammalian DNA virus in a non-mammalian host cell expressing the gene required for replication that is deleted or inactivated in the non-mammalian DNA virus.

Yet another embodiment of the present invention, provides a composition comprising a genome of a non-mammalian DNA virus comprising an exogenous gene encoding a fluorescent sensor molecule operably linked to a mammalian promoter. The non-mammalian DNA virus in certain illustrative embodiments, is a baculovirus. The composition can be a viral particle. In another embodiment, provided herein are methods that utilize the composition, for example the viral particle to express the fluorescent sensor molecule in a mammalian cell. The fluorescent sensor molecule, in illustrative examples, is a fluorescent protein. In certain embodiments, the exogenous gene encodes a biosensor comprising a fluorescent protein.

Another embodiment includes a method for providing a mammalian cell reporter vector, comprising, offering the mammalian cell reporter vector for sale to a customer along with the right to use the mammalian cell reporter but not the right to replicate the mammalian cell reporter vector, wherein the mammalian cell reporter vector is a non-mammalian DNA virus comprising a mammalian promoter or mammalian-active promoter, operatively linked to a reporter gene.

A further embodiment of the invention is a method for selling a composition comprising a genome of a non-mammalian DNA virus wherein a gene required for replication in a non-mammalian host cell is deleted or inactivated and further comprising an exogenous gene operably linked to a mammalian promoter by presenting to a customer an identifier that identifies the composition and providing to the customer access to a purchase function for purchasing the composition.

Another embodiment of the invention is an ordering system for selling a composition comprising a genome of a non-mammalian DNA virus wherein a gene required for replication in a non-mammalian host cell is deleted or inactivated and further comprising an exogenous gene operably linked to a mammalian promoter the system comprising an input function for identifying a desired product, and a purchasing function for purchasing a desired product that is identified. In certain illustrative embodiments, the ordering system is a computer-based ordering system. Thus instructions for performing the functions of the ordering system are provided in computer readable form on a computer storage medium, such as a storage drive, such as a hard drive on a server or a personal computer.

Other embodiments of the invention encompass methods for selling a composition comprising a genome of a non-mammalian DNA virus wherein a gene required for replication in a non-mammalian host cell is deleted or inactivated and further comprising an exogenous gene operably linked to a mammalian promoter by presenting to a customer an input function of a telephonic ordering system, and/or presenting to a customer a data entry field or selectable list of entries as part of a computer system, wherein the composition is identified using the input function. A further embodiment of the invention is a method wherein the input function is part of a computer system such as displayed on one or more pages of an internet site, the method further comprising presenting to the customer an on-line purchasing function, such as an online shopping cart, wherein using the purchasing function the customer purchases the identified composition. In some embodiments the method further comprises shipping the purchased composition to the customer.

Another embodiment is a commercial product comprising a genome of a non-mammalian DNA virus wherein a gene required for replication in a non-mammalian host cell is deleted or inactivated and further comprising an exogenous gene operably linked to a mammalian promoter. The exogenous gene is, for example, a genetically encoded reporter molecule, such as a fluorescent protein, or a sensor comprising a fluorescent protein.

Further embodiments include an ordering system for selling a composition comprising a genome of a non-mammalian DNA virus wherein a gene required for replication in a non-mammalian host cell is deleted or inactivated and further comprising an exogenous gene operably linked to a mammalian promoter the system comprising an input function for identifying a desired product, and a purchasing function for purchasing a desired product that is identified. In certain illustrative embodiments, the ordering system is a computer-based ordering system.

Other embodiments include a viral particle comprising a genome of a non-mammalian DNA virus, wherein a gene required for replication in a non-mammalian host cell is deleted or inactivated and further comprising an exogenous gene operably linked to a mammalian promoter.

A still further embodiment is a kit comprising, a viral particle or a vessel comprising a viral particle comprising a genome of a non-mammalian DNA virus, wherein a gene required for replication in a non-mammalian host cell is deleted or inactivated and further comprising an exogenous reporter gene operably linked to a mammalian promoter. In certain embodiments, the exogenous reporter gene encodes a fluorescent or luminescent protein, or a cell sensor comprising a fluorescent or luminescent protein. The kit can further include a vessel comprising a fluorescence or luminescnece enhancing agent. The kit may also provide a vessel comprising a solubilizing agent for preparing stock solutions of the enhancing agent.

Another embodiment is a method for expressing an exogenous gene in a mammalian cell comprising contacting a non-mammalian DNA virus with the mammalian cell such that the non-mammalian DNA virus infects the mammalian cell and the exogenous gene is expressed, wherein the non-mammalian DNA virus comprises a genome comprising an exogenous gene operably linked to a mammalian promoter, and wherein the exogenous gene encodes a reporter molecule, biosensor, cameleon or other fluorescent sensor. In certain aspects, the reporter molecule is operably linked to an intracellular localization sequence, such as a sequence that targets the reporter molecule to a subcellular location such as an organelle.

Other embodiments include methods for selling a recombinant biosensor, comprising presenting to a customer an identifier that identifies the recombinant biosensor and providing to the customer access to a purchase function for purchasing the recombinant biosensor, wherein the recombinant biosensor comprises a genome of a non-mammalian DNA virus comprising a coding sequence encoding the recombinant biosensor linked to a mammalian promoter.

A further embodiment is a method for detecting a biomolecule, comprising contacting a non-mammalian DNA virus with the mammalian cell such that the non-mammalian DNA virus infects the mammalian cell and the exogenous gene is expressed, wherein the non-mammalian DNA virus comprises a genome comprising an exogenous gene operably linked to a mammalian promoter, and wherein optionally a gene required for replication of the non-mammalian DNA virus in a non-mammalian host cell is deleted from the genome or inactivated, and wherein the exogenous gene encodes a biosensor that binds the biomolecule; and detecting the expressed biosensor, wherein the biosensor undergoes a detectable change upon binding to the biomolecule.

Another embodiment is a method for expressing an exogenous gene in a mammalian cell comprising contacting a non-mammalian DNA virus with the mammalian cell such that the non-mammalian DNA virus infects the mammalian cell and the exogenous gene is expressed, wherein the contacting is performed by a user not having a legal right to replicate the non-mammalian DNA virus.

Another embodiment provided herein, is a method for expressing two or more exogenous genes in a mammalian cell comprising contacting a non-mammalian DNA virus with the mammalian cell such that the non-mammalian DNA virus infects the mammalian cell and the exogenous genes are expressed, wherein the two or more exogenous genes encode a genetically-encoded sensor and a second messenger system protein. For example, in this embodiment, the non-mammalian DNA virus can encode an ion channel protein and a second messenger sensor, such as a calcium sensor. In a related embodiment, the present invention provides a non-mammalian DNA virus that encodes two or more exogenous genes operably linked to a mammalian promoter, wherein the two genes encode a genetically-encoded sensor and a second messenger system protein. The two or more genes can be expressed as part of a polycistron.

The invention is useful for expressing an exogenous gene(s) in a mammalian cell (e.g., HepG2 or CHO cells). This method can be employed in the manufacture of proteins to be purified, such as proteins which are used pharmaceutically (e.g., insulin) or to express genetically encoded reporter molecules such as fluorescent proteins. Virtually any fluorescent protein can be used in the present invention. For example, the fluorescent protein can be an Aequorea fluorescent protein (See e.g., Tsien, Annu. Rev. Biocehem. 1998, 67:509-44), or a mutant of an Aequorea fluorescent protein that is, for example, 75%, 80%, 85%, 90%, 95%, 97%, 98%, 99% or 100% identical to Aequorea victorea GFP. The fluorescent protein is typically a recombinant fluorescent protein. In certain embodiments that involve a sensor, two fluorescent proteins can be included in the sensor, typically where the two fluorescent proteins form a FRET pair, which can be encoded as a fusion protein. Fluorescent proteins and GFP variants that can be used in the present invention include, but are not limited to, green fluorescent protein, including Emerald GFP, Topaz, sapphire, CFP, Cycle 3, orange fluorescent protein (OFP), yellow fluorescent protein (YFP), such as the Venus mutant, red fluorescent protein, blue fluorescent protein. Furthermore, in certain embodiments, especially where a cell sensor is included, a circularly permuted version of a fluorescent protein can be utilized. In other examples, the fluorescent protein is a Renilla fluorescent protein, such as a wild-type, a recombinant, or a mutant, recombinant fluorescent protein.

The non-mammalian viral expression system of the invention offers several advantages. The invention allows for de novo expression of an exogenous gene; thus, detection of the exogenous protein (e.g., β-galactosidase) in an infected cell represents protein that was actually synthesized in the infected cell, as opposed to protein that is carried along with the virus aberrantly. The non-mammalian viruses used in the invention are not normally pathogenic to humans; thus, concerns about safe handling of these viruses are minimized. Similarly, because the majority of naturally-occurring viral promoters are not normally active in a mammalian cell, production of undesired viral proteins is inhibited. For example, PCR-based experiments indicate that some viral late genes are not expressed. In addition, the use of serum-free media eliminates a significant expense faced by users of mammalian viruses. Certain non-mammalian viruses, such as baculoviruses, can be grown to a high titer (i.e., 10⁸ pfu/ml). Generally, virus genomes are large (e.g., the baculovirus genome is 130 kbp); thus, viruses used in the invention can accept large exogenous DNA molecules. In certain embodiments, the invention employs a virus whose genome has been engineered to contain an exogenous origin of replication (e.g., the EBV oriP). The presence of such sequences on the virus genome allows episomal replication of the virus, increasing persistence in the cell. Where the invention is used in the manufacture of proteins to be purified from the cell, the invention offers the advantage that it employs a mammalian expression system. Accordingly, one can expect proper post-translational processing and modification (e.g., glycosylation) of the gene product.

The present invention can be practiced in certain illustratative embodiments with recombinant baculoviruses that contain an insertionally inactivated or deleted gp64 efp gene, a gene that encodes a protein essential for viral infectivity and propagation in cell culture and in animals. To generate the virus the GP64 EFP protein can be supplied in trans, from a stably transfected cell line. Homologous recombination can be used to generate inactivated gp64 efp genes in the context of an otherwise wild type AcMNPV baculoviruses. For generating the stably transfected cell line, a heterologous gp64 efp gene (derived from a different baculovirus, OpMNPV) can be selected. Viruses containing either a) an insertional inactivation of the gp64 efp ORF, or b) a complete deletion of the gp64 ORF can be generated by this method.

These and other advantages of the present invention will become apparent from the following detailed description.

### BRIEF DESCRIPTION OF THE DRAWINGS

A more complete understanding of the present invention may be derived by referring to the detailed description and claims when considered in conjunction with the following figures.

FIG. 1 provides the construction of the gp64-lacZ transfer vector pAcgp64Z-Δ-Nco. Plasmid names are indicated for each construct (diagrams not to scale).

FIG. 2 A diagram depicting how Sf9 cells may be stably transfected with a gp64.

FIG. 3 A diagram depicting the pFB CMV RSV (A) plasmid.

FIG. 4 The sequence of the pFB CMV RSV (A) plasmid.

### DETAILED DESCRIPTION OF THE INVENTION

The present invention, in certain embodiments, provides compositions and methods for expressing exogeneous nucleic acid sequences in mammalian cells using non-mammalian DNA viruses, for example baculoviruses. In illustrative examples, the non-mammalian DNA viruses are unable to replicate in the viruses' natural non-mammalian host cell. Furthermore, in illustrative examples, the compositions and methods express in mammalian cells, reporter molecules, especially fluorescent proteins, or even fluorescent protein biosensors. The invention further provides methods for the commercial sale of these compositions.

A wide variety of non-mammalian DNA viruses may be used in the present invention. By “non-mammalian” DNA virus is meant a virus which has a DNA genome (rather than RNA) and which is naturally incapable of replicating in a vertebrate, and specifically a mammalian, cell. Included are insect viruses, plant viruses, and fungal viruses. By “insect” DNA virus is meant a virus which has a DNA genome and which is naturally capable of replicating in an insect cell (e.g., Baculoviridae, Iridoviridae, Poxyiridae, Polydnaviridae, Densoviridae, Caulimoviridae, and Phycodnaviridae). Viruses which naturally replicate in prokaryotes are excluded. Examples of viruses that are useful in practicing the invention are described in Tablel of U.S. Pat. No. 6,238,914. In many embodiments of the invention, an invertebrate baculovirus such as Autographa californica multiple nuclear polyhedrosis virus (AcMNPV) is used.

In certain aspects, the viral vector is not replicable in its natural host cell. By “natural host cell” is meant that the cell in which the non-mammalian DNA virus is normally replicated in and which has not been modified by recombinant DNA technology methods. In order to obtain a virus that is unable to replicate in its natural host cell, the function of a gene that is required for infectivity or replication is either deleted or deactivated. In order to produce the resulting non-replicative virus, a complimentary host cell is developed which expresses the deleted or deactivated gene thereby allowing the non-replicative virus to propagate.

The invention is not limited to the inactivation or deletion of any particular gene so long as the inactivated or deleted gene is required for replication or infectivity of the virus and the function of the gene can be supplied in trans by a host cell modified to express the inactivated or deleted gene. Other genes that may prove useful for generating viruses with null-mutations and similar rescue strategies include other genes that are essential or important for viral structure, replication or propagation in cell culture. Such genes may include capsid protein genes (vp 39, p80/87, p24 or p78/83), other as yet uncharacterized envelope protein genes form the budded form of the virus or essential regulatory genes such as ie-1, ie-N, (ie-2), and lef genes. Other mutations that affect the expression of these genes may be possible. For example deletion or inactivation of promoters or other control elements for essential genes could accomplish the same purpose. Previous studies of the GP64 EFP protein in baculoviruses demonstrated that some anti-GP64 antibodies are capable of neutralizing infectivity of the virus. In some embodiments of the invention the gp64 efp gene of a baculovirus is inactivated by homologous recombination and the GP64 EFP protein is supplied in trans from a stably transfected cell line.

Because a very strong selection pressure for regenerating a gp64 EFP+ virus may result in recombination between the virus and the gp64 efp gene within the cell line, the following strategies can be used:

For generating the stably transfected cell line, a heterologous gp64 efp gene (derived from a different baculovirus, OpMNPV) can be selected.

b) Recombinant vAc^{64Z} virus stocks are screened by restriction analysis, Western blots and PCR for significant levels of any revertant virus.

c) A lacZ marker gene is fused in-frame with the wt AcMNPV gp64 efp gene, and analyses of the “loss-of-function” phenotype of the recombinant virus is based on detection of the β-galactosidase marker.

d) A second vAc^{64Z} virus with the gp64 efp ORF completely deleted is generated. The analysis of only cells expressing the lacZ marker gene insures that viruses carrying the inactivated gp64 efp gene are exclusively analyzed.

Generation of Transfected Cell Lines

An initial step in making certain compositions of the invention is to produce cell lines that constitutively express the inactivated or deleted gene of the virus. From the above discussion it is clear that the invention can be practiced using any virus, in illustrative embodiments, a non-mammalian DNA virus, with an inactivated or deleted gene necessary for viral infectivity or replication. Solely for the purposes of simplifying the explanation, the following discussion will use the gp64 efp gene as a non-limiting example of a gene to be inactivated and baculovirus as a non-limiting example of a non-mammalian DNA virus. The use of these examples should not in any way be construed as limiting the broad scope of the disclosure. Because previous studies using anti-GP64 EFP antibodies suggested that GP64 EFP might be an essential component of budded virions, a strategy in which the GP64 EFP protein is provided in trans, to complement the inactivation of the gp64 efp gene in the virus is used. To provide GP64 EFP in trans, a stably transfected cell line that constitutively expresses the OpMNPV GP64 EFP protein is generated by transfection with plasmids encoding gp64 EFP and the bacterial neomycin resistance gene, followed by selection for G418 resistance.

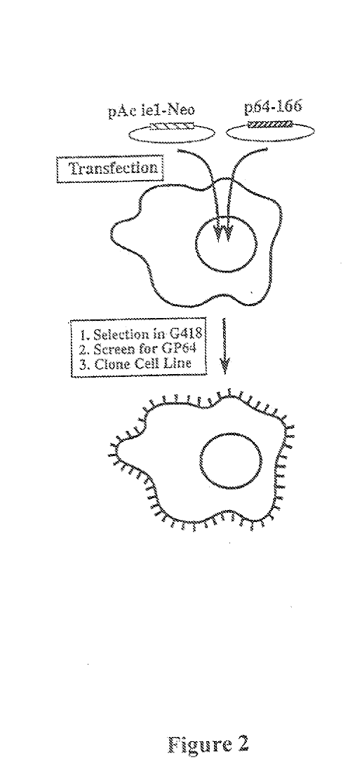

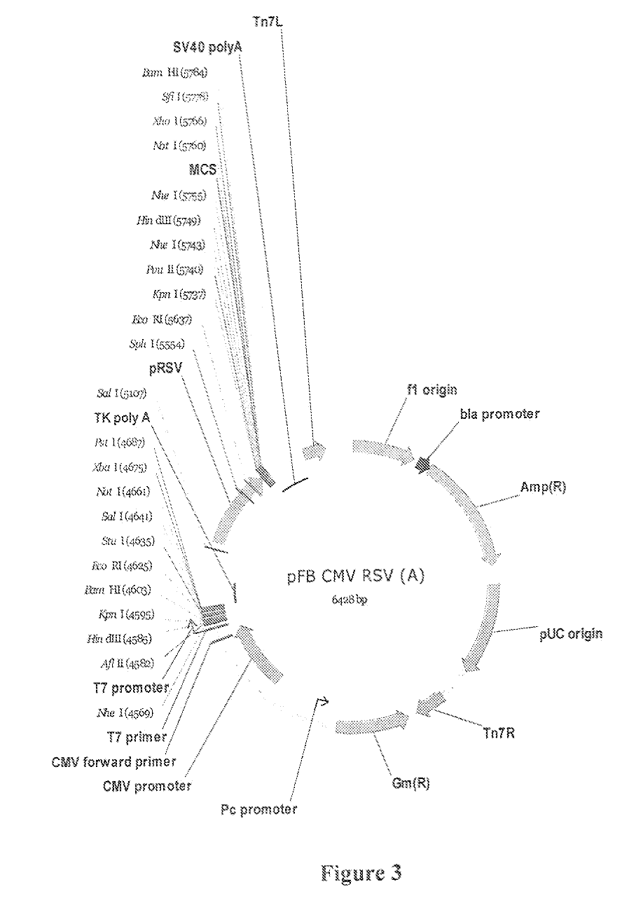

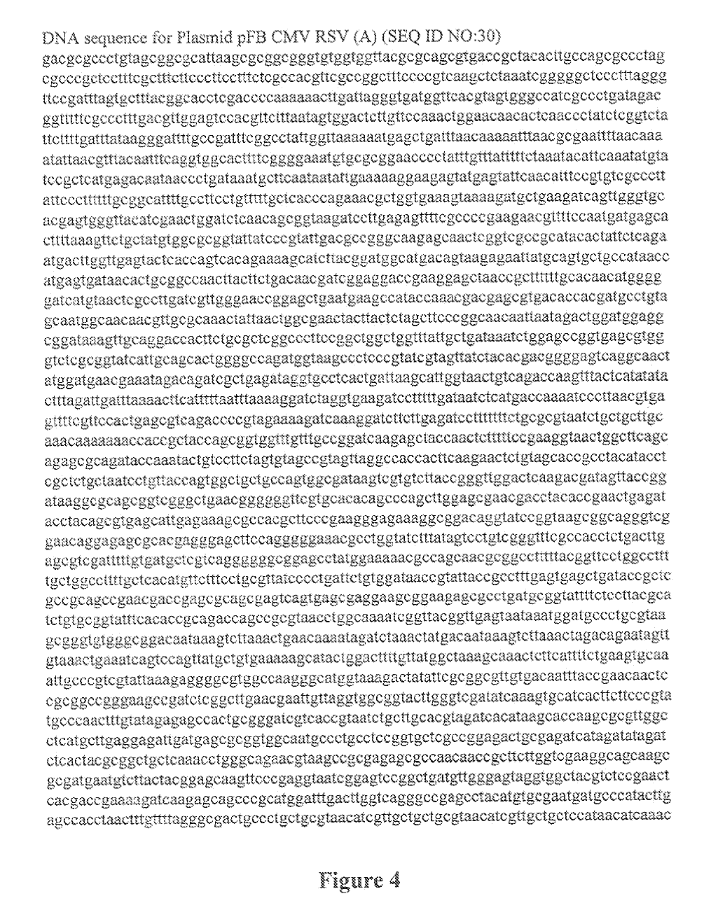

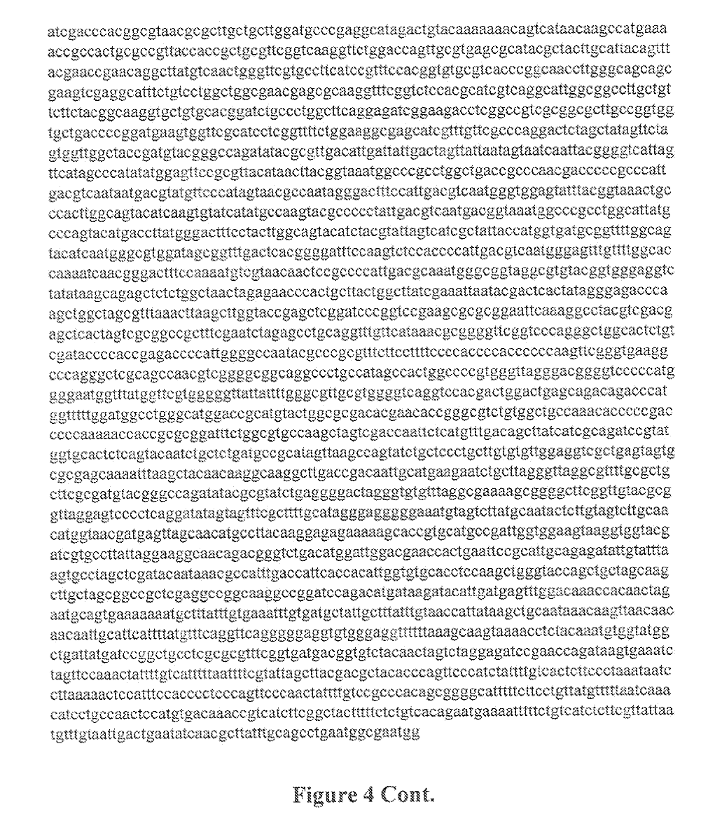

For production of the stably transfected cell lines (and for propagation of AcMNPV), Spodoptera frugiperda Sf9 cells are cultured in TNM-FH complete medium containing 10% fetal bovine serum at 27° C. To express the OpMNPV gp64 EFP, a gp64 EFP expression plasmid (p64-166) that contains the OpMNPV gp64 efp ORF under the control of an OpMNPV gp64 efp early promoter construct is used. (See FIG. 2.)

The second plasmid (pAc ie1-Neo) encodes a bacterial neomycin resistance gene under the control of the AcMNPV ie1 promoter, and is constructed using the approach described by Jarvis et al. (1990). Transfected Sf9 cells that are resistant to G418 are selected, and isolated cell lines established. Transfection and G418 selection is performed essentially as described previously by Jarvis et al. (1995). Briefly, Sf9 cells are plated at a density of 1×10⁶ cells per well (34 mm diameter). The cells are transfected with 2 μg p64-166 plasmid plus 1 μg pAc ie 1-Neo using calcium phosphate precipitation. One day after transfection, the cells are replated at low density in 75 cm² flasks and maintained for 2 weeks in TNM-FH complete media containing 1 mg/ml G418 (Geneticin, GIBCO). During this period, mock-transfected Sf9 control cells can be used as a control. The G418-resistant transfected cells are replated in TNM-FH complete medium (lacking G418) at low density. Single colonies are isolated and transferred to individual wells of a 24 well plate. Isolated lines are screened for gp64 EFP expression by cell-surface staining of paraformaldehyde-fixed cells using MAb AcV5 and an alkaline phosphatase-conjugated goat-anti-mouse secondary antibody. Isolated lines are also screened for GP64 EFP fusion activity using a syncytium formation assay.

Generation of a gp64 EFP-Null AcMNPV Baculovirus

Viral DNA used for the generation of recombinant viruses is prepared from the E2 strain of AcMNPV by standard methods. For production of budded virus (BV) stocks and occlusion bodies of the wild type and recombinant viruses, cells (Sf9 or Sf9^{OP64-6}) are infected at a multiplicity of infection (MOI) of 0.1 and incubated at 27° C. for 5 to 7 days. Supernatants are harvested and titred by end-point dilution. The recombinant AcMNPV virus lacking GP64 expression is titred on the Sf9^{OP64-6} cells, and the wild type AcMNPV virus and recombinant virus vAc^{hsZ} are titred on Sf9 cells. Occlusion bodies are purified from infected cells by sequential washing with 0.5% SDS, 0.5M NaCl and distilled water. For analysis of budded virion structural proteins, budded virions are isolated from viral stocks by pelleting through a 25% sucrose pad followed by centrifugation on 25%-60% sucrose gradients. The budded virus band is collected, diluted in PBS pH 6.2, pelleted and resuspended in SDS lysis buffer for SDS-PAGE on 10% acrylamide gels.

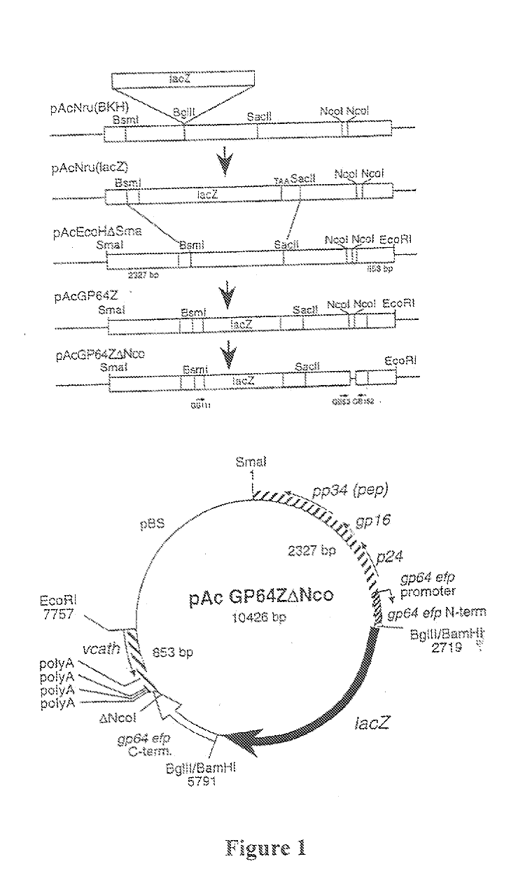

To inactivate the gp64 efp gene in AcMNPV, insertional mutagenesis can be used. The AcMNPV gp64 efp gene is inactivated by inserting the bacterial lacZ ORF (in frame) into the gp64 efp ORF after codon 131 as shown in FIG. 1. Although the lacZ ORF contains a translation termination codon, a frame-shift mutation after codon 452 of gp64 efp can also be created, by removing a 54 bp NcoI restriction fragment. This frame shift results in a translation termination codon immediately upstream of the gp64 EFP transmembrane domain and insures inactivation of the gp64 efp gene. The gp64-lacZ fusion gene is then cloned into a transfer vector containing sequences flanking the gp64 efp locus in AcMNPV. Recombination of this transfer vector into the gp64 efp locus of wild type AcMNPV results in a virus wild type for all genes except gp64 efp.

To construct the transfer vector for allelic replacement of the gp64 efp locus of the AcMNPV genome, the 4718 bp EcoRI-SmaI fragment (corresponding to nucleotides 107,326 to 112,043 from the EcoRI H fragment) of AcMNPV strain E2 is cloned into the pBS vector (Stratagene) to generate the plasmid pAcEcoHδSma. This plasmid contains 2327 bp upstream of the gp64 efp ORF, the gp64 efp ORF, and 853 bp downstream of the gp64 efp ORF. To disrupt the AcMNPV gp64 efp gene by insertional mutagenesis, an in-frame fusion between gp64 EFP and the Escherichia coli lacZ gene in an AcMNPV gp64 EFP expression plasmid pAcNru(BKH) is generated. The pAcNru(BKH) expression plasmid contains an 18 bp in-frame linker encoding unique BglII, KpnI and HindIII restriction sites, inserted at the NruI restriction site within the gp64 efp ORF of plasmid p166B+1 Ac Spe/Bgl. A 3072 bp BamHI fragment containing the lacZ ORF (derived from pMC1871) is subcloned into BglII digested pAcNru(BKH). The resulting construct (pAcNru(lacZ)) contains a gp64-lacZ fusion after codon 131 of gp64 efp, and the fusion gene open reading frame terminates at the end of the lacZ insertion. The 3447 bp BsmI/SacII fragment of pAcNru(lacZ) (containing the lacZ cassette and the flanking portions of gp64 efp) is subcloned into BsmI/SacII digested pAcEcoHδSma, to generate plasmid pAcgp64Z. Finally, to ensure inactivation of the gp64 efp gene, the downstream portion of the gp64 efp ORF is truncated by digesting pAcgp64Z with NcoI, removing the resulting 54 bp NcoI-NcoI fragment, then blunting and religating to generate pAcgp64ZδNco. This deletion results in a frame shift mutation and terminates the gp64 efp open reading frame after codon 452, 30 codons upstream of the predicted transmembrane domain.

FIG. 1 shows the construction of the gp64-lacZ transfer vector pAcgp64Z-Δ-Nco. Plasmid names are indicated for each construct (diagrams not to scale). Small arrows below the bottom diagram indicate the locations of primers (SEQ ID: 1 (GB 111), SEQ ID:3 (GB 53), SEQ ID:2 (GB 152)) used for PCR analysis. A map of the gp64-lacZ transfer vector pAcgp64ZΔNco is shown. The transfer vector contains 2327 bp of flanking sequences upstream of the gp64 efp ORF, and 853 bp of flanking sequences downstream of the wild type gp64 efp stop codon. Also indicated are the locations and orientations of genes flanking the gp64 efp locus (vcath, p24, gp 16 and pp 34 (pep)).

Recombinant viruses are generated using standard protocols, by co-transfecting viral DNA from wild-type AcMNPV strain E2 and pAcgp64ZΔNco plasmid DNA into the gp64 EFP expressing SF9^{OP64-6} cells. A recombinant virus (vAc^{64Z}) is isolated from culture supernatant by plaque purification on SF9^{OP64-6} cells, using X-gal in the agarose overlay to identify the recombinant plaques.

The structure of the gp64 efp locus in vAc^{64Z} can be analyzed by PCR amplification and restriction mapping. DNA is extracted from infected Sf9 cells at 24 h pi, or viral DNA is isolated from BV pelleted through a 25% sucrose cushion. For PCR analysis, the following primers homologous to the 5′ and 3′ ends of the gp64 efp ORF can be used:

SEQ ID:1 (GB 111),
5′-GAGCTGATCGACCGTTGGGG-3′;
and
SEQ ID:2 (GB 152),
5′-CGGTTTCTGATCATACAGTACA-3′.

To verify the presence of the NcoI deletion in vAc64Z, PCR amplification is performed using primers flanking the deletion site:

SEQ ID:3 (GB 53),
5′-CCAGCGGCTGGTCGTTTATCGCCC-3′;
and
SEQ ID:2 (GB 152),
5′-CGGTTTCTGATCATACAGTACA-3′.

To generate recombinant viruses defective for gp64 EFP, transfections, recombination and viral growth is carried out in the Sf90P64-6 cell line. A recombinant virus (vAc^{64Z}) can be isolated by plaque purification on SF9^{OP64-6} cells, using X-gal in the plaque overlay to detect β-galactosidase expression. Although the gp64 efp promoter is active in both early and late phases of infection, β-galactosidase activity is not detectable before 5-7 days post infection, unless the plaque assay plates are subjected to a freeze-thaw cycle to disrupt the infected cells. This suggests that active gp64-β-galactosidase fusion protein is not secreted into the medium, despite the presence of the gp64 EFP signal peptide in the GP64-β-galactosidase fusion protein.

To confirm the location of the gp64-lacZ insertion in vAc^{64Z}, the gp64 efp locus of the vAc^{64Z} recombinant virus can examined by PCR amplification using primers complementary to the 5′ and 3′ ends of the gp64 efp gene. As control templates for PCR, a plasmid containing the wild-type AcMNPV gp64 efp gene (negative control), DNA from cells infected with wild type AcMNPV, or plasmid DNA of the transfer vector pAcgp64ZΔNco (positive control) can be used. Amplification from the plasmid containing the wild type gp64 efp gene or AcMNPV viral DNA will result in a 1.17 kb product, as predicted from the sequence. Amplification from the pAcgp64ZΔNco transfer vector or DNA from cells infected by vAc^{64Z} will result in a single 4.22 kb product, as predicted from allelic replacement of the gp64 efp locus by the gp64-lacZ fusion gene. PCR analysis (using primers SEQ ID:3 (GB 53) and SEQ ID:2 (GB 152)) can also be used to verify that the recombinant virus vAc^{64Z} lacks the NcoI fragment deleted from the downstream region of the gp64 efp ORF. The structure of the recombinant virus vAc^{64Z} can also examined by restriction enzyme digestion of viral genomic DNA.

To verify that the gp64 efp gene is inactivated, vAc^{64Z} infected Sf9 cells can be examined by ECL-Western blot analysis. Cells are infected with either vAC^{64Z} or wild type AcMNPV, and cell lysates are prepared at 24 and 48 h pi. Replicate blots are probed with monoclonal antibodies specific to β-galactosidase, GP64 EFP, or P39 capsid protein. A replicate blot is also probed with a mixture of all three antibodies. Expression of β-galactosidase is expected to be detected only in cells infected with vAc^{64Z} and not in cells infected with wild type AcMNPV.

The presence of OpMNPV GP64 EFP on budded virions produced by vAc^{64Z} infected Sf9^{OP64-6} cells can be demonstrated by examining by Western blot analysis of budded virions purified from tissue culture supernatants by sucrose gradient centrifugation. As a control, budded virions are purified from tissue culture supernatants of AcMNPV infected Sf9 cells. Replicate blots are probed with either a) a monoclonal antibody that reacts only with the OpMNPV GP64 EFP, b) a monoclonal antibody that reacts with GP64 EFPs of both OpMNPV and AcMNPV, or c) a monoclonal antibody that reacts with the P39 capsid protein. GP64 EFP is expected to be detected in purified budded virions of both AcMNPV and vAc^{64Z} by monoclonal antibody AcV5 (which cross reacts with GP64 EFPs of both AcMNPV and OpMNPV).

The invention can be used with other essential genes, so long as there is a means for generating sufficient amounts of the null virus for the cloning system. The gp64 gene is an excellent choice because the gene is essential for production of infectious virions.

Other baculoviruses are suitable for use with the invention. The gp64 gene has been isolated and sequenced in a number of baculoviruses. For example, non-replicative viruses could be created from the teachings herein and information available in the literature for the following systems: Orgyia pseudotsugata MNPV (OpMNPV), Trichoplusia ni SNPV (TnSNPV), Lymantria dispar MNPV (LdMNPV), Choristoneura fumiferana MNPV (CfMNPV), Bombyx mori NPV (BmNPV), and other baculoviruses. To produce recombinant baculoviruses of the invention, a bacmid system can be used (See e.g., U.S. Pat. No. 5,348,886, incorporated herein by reference in its entirety). Briefly, infectious recombinant baculoviruses can be produced in bacteria using a baculovirus shuttle vector (bacmid) constructed to contain a low-copy-number bacterial replicon, a selectable drug resistance marker, and a preferred attachment site for a site-specific bacterial transposon, inserted into a nonessential locus of the baculovirus genome. This shuttle vector can replicate in E. coli as a plasmid and is stably inherited and structurally stable after many generations of growth. Bacmid DNA isolated from E. coli can be used to infect susceptible lepidopteran insect cells. DNA segments containing a promoter, such as a mammalian promoter, or multiple promoters, driving expression of one or more foreign genes that are flanked by the left and right ends of the site-specific transposon can transpose to the attachment site in the bacmid propagated in E. coli when transposition functions can be provided in trans by a helper plasmid. The foreign gene can be expressed when the resulting composite bacmid is introduced into cells, such as mammalian cells.

A variety of exogenous genes may be used to encode gene products such as proteins, antisense nucleic acids (e.g., RNAs), catalytic RNAs, biosensor or reporter proteins. By “exogenous” gene or promoter is meant any coding region or promoter that is not normally part of the non-mammalian DNA virus (e.g., baculovirus) genome. Such genes include those genes which normally are present in the mammalian cell to be infected; also included are genes which are not normally present in the mammalian cell to be infected (e.g., related and unrelated genes of other cells or species). If desired, the gene product (e.g., protein or RNA) may be purified from the cell. Thus, the invention can be used in the manufacture of a wide variety of proteins that are useful in the fields of biology and medicine. Suitable reporter proteins include, but are not limited to, fluorescent proteins and cameleon chimeras of fluorescent proteins (Miyawaki et al. Nature 1997, vol. 388(6645):882-7 and U.S. Pat. No. 5,998,204 incorporated herein by reference in their entirety). Biosensor, reporter, cameleon or fluorescent sensor molecules may be used to detect pH, the presence of ions including metal ions and other biomolecules by undergoing a detectable change when coming in contact with the changed pH, ion or biomolecule. Exemplary biosensors include, but are not limited to, cameleon calcium sensor (U.S. Pat. No. 5,998,204: Fluorescent protein sensors for detection of analytes, incorporated by reference in its entirety), cAMP sensor (WO06054167A2: BIOSENSOR FOR DETECTION OF CAMP LEVELS AND METHODS OF USE), IP3 sensor, redox sensor, pH sensor, AKAR kinase sensor, BFP, CFP, GFP, YFP, OFP, and RFP sensors.

The exogenous gene is positioned for expression so that it is expressed in the mammalian cell. This generally means that the exogenous gene is operably linked to a mammalian promoter. By “positioned for expression” is meant that the DNA molecule which includes the exogenous gene is positioned adjacent to a DNA sequence which directs transcription and, if desired, translation of the DNA and RNA (i.e., facilitates the production of the exogenous gene product or an RNA molecule). By “operably linked” is meant that a gene and a regulatory sequence(s) (e.g., a promoter) are connected in such a way as to permit gene expression when the appropriate molecules (e.g., transcriptional activator proteins) are bound to the regulatory sequence(s). By “promoter” is meant a minimal sequence sufficient to direct transcription. Also useful in the invention are those promoters which are sufficient to render promoter-dependent gene expression controllable for cell-type specificity, cell-stage specificity, or tissue-specificity (e.g., liver-specific promoters), and those promoters which are inducible by external signals or agents; such elements can be located in the 5′ or 3′ regions of the native gene. Promoters which may be used with the invention include, but are not limited to, tk, CMV, RSV, EF1, SV40, Ubi-c, human H1, human U6, mouse H1, Pmin, and EF-1a promoters.

Established methods for manipulating recombinant viruses may be incorporated into these new methods for expressing an exogenous gene in a mammalian cell. The genome of the non-mammalian DNA virus can be engineered to include one or more genetic elements, such as a promoter of a long-terminal repeat of a transposable element or a retrovirus (e.g., Rous Sarcoma Virus); an integrative terminal repeat of an adeno-associated virus; and/or a cell-immortalizing sequence, such as the EBNA-1 gene of Epstein Barr Virus (EBV). If desired, the genome of the non-mammalian DNA virus can include an origin of replication which functions in a mammalian cell (e.g., an EBV origin of replication or a mammalian origin of replication). Origins of replication derived from mammalian cells have been identified (Burhans et al., 1994, Science 263:639-640). Other origins of replication, such as the Epstein-Barr Virus oriP, can also facilitate maintenance of expression in the presence of appropriate trans-acting factors, such as EBNA-1.

The genome of the non-mammalian DNA virus used in the invention can include a polyadenylation signal and an RNA splicing signal positioned for proper processing of the product of the exogenous gene. In addition, the virus may be engineered to encode a signal sequence for proper targeting of the gene product.

Intracellular targeting sequences are known that can target a fusion protein to any one of many specific subcellular locations. For example, intracellular targeting sequences are known that target proteins to the nucleus (Dingwall, C., 1991, TIBS 16(12) 478-81 and SEQ ID NO:4, PSKKKRKV), the mitochondria (Hanson, G., 2004 J. Biol. Chem. 279(13) 13044-53 and SEQ ID NO:5, MRKMLAAVSRVLSGASQKPASRVLVASRN), the peroxisome (Gould, S J, 1989, JCB 108: 1657-64), the endoplasmic reticulum (Fliegel, L., 1989, J. Biol. Chem., 264(36) 21522-8 and SEQ ID NO:6 MLLPVPLLLGLLGLAAA), the plasma membrane (Kabouridis, P S., 1997, EMBO 16(16) 4983-98 and SEQ ID NO:7, MGCVCS), the golgi apparatus (Storrie, B., 1998 JCB 143(6) 1505-21 and SEQ ID NO:8, MRRRSRMLLCFAFLWVLGIAYYMYSGGGSALAGGAGGGAGRKEDWNEIDPIKK KDLHHSNGEEKAQSMETLPPGKVRWPDFNQEAYVGGTMVRSGQDPYARNKFN QVESDKLR), the cytoplasm (Chevalier, S A, 2005, BMC-R 2(70) 1-11 and SEQ ID NO:9, KRLEELLYKMFLHT), the nuclear envelope (Zhang, Q., 2001, JCS 114: 4485-98 and SEQ ID NO:10, RGFLFRVLRAALPLQLLLLLLIGLACLVPMSEEDYSCALSNNFARSFHPMLRYTN GPPPL). Additional targeting sequences are described by Watson, 1984, Nucleic Acids Research, 12:5145-5164 (incorporated by reference in its entirety). Intracellular targeting sequences are typically covalently attached to the amino terminus of a fusion protein by being expressed from an expression vector that includes nucleic acids encoding the intracellular targeting sequence in frame with a protein of interest, such as a fluorescent protein such as GFP, OFP, CFP, RFP, YFP, etc. Exemplary targets include, but are not limited to GPCRs, kinases, nuclear receptors, ion channels, G-proteins, transporters, transcription factors, glycosidases/glycosyltransferases, phosphodiesterases, proteases, and protein phosphatases.

Where cell-type specific expression of the exogenous gene is desired, the genome of the virus can include a cell-type-specific promoter, such as a liver cell-specific promoter. Examples of suitable promoters include the RSV LTR, the SV40 early promoter, the CMV IE promoter, the adenovirus major late promoter, and the Hepatitis B promoter. In addition, promoters which are cell-type-specific, stage-specific, or tissue-specific can be used. For example, several liver-specific promoters, such as the albumin promoter/enhancer, have been described (see, e.g., Shen et al., 1989, DNA 8:101-108; Tan et al., 1991, Dev. Biol. 146:24-37; McGrane et al., 1992, TIBS 17:40-44; Jones et al., J. Biol. Chem. 265:14684-14690; and Shimada et al., 1991, FEBS Letters 279:198-200). Where the invention is used to treat a hepatocellular carcinoma, an α-fetoprotein promoter is particularly useful. This promoter is normally active only in fetal tissue; however, it is also active in liver tumor cells (Huber et al., 1991, Proc. Natl. Acad. Sci. 88:8039-8043). Accordingly, an α-fetoprotein promoter can be used to target expression of a liver-cancer therapeutic to liver tumor cells. Further examples include α-1-antitrypsin, pyruvate kinase, phosphenol pyruvate carboxykinase, transferrin, transthyretin, α-fetoprotein, α-fibrinogen, or β-fibrinogen. Alternatively, a hepatitis B promoter may be used. If desired, a hepatitis B enhancer may be used in conjunction with a hepatitis B promoter. Preferably, an albumin promoter is used. Other preferred liver-specific promoters include promoters of the genes encoding the low density lipoprotein receptor, α2-macroglobulin, α1-antichymotrypsin, α2-HS glycoprotein, haptoglobin, ceruloplasmin, plasminogen, complement proteins (C1q, C1r, C2, C3, C4, C5, C6, C8, C9, complement Factor I and Factor H), C3 complement activator, β-lipoprotein, and α1-acid glycoprotein.

Essentially any mammalian cell can be used to express the exogenous gene; in some embodiments the mammalian cell is a 143TK cell, Astroglioma U373MG cell, Bone marrow fibroblasts, Bone marrow stem cells (bMSC), CHP212 cells, C₂-C₁₂ cells, Coronary artery endothelia cells (hCEC), DLS-1 cells, Embryonic lung fibroblasts, FLC4 cells, HEK 293 cells, HeLa cells, HepG2 cells, Huh7 cells, HUVEC cells, IMR32 cells (CCL-127 neuroblastoma), KATO-III cells (HTB-103), Keratinocytes, MG63 cells, RC5 cells, CRL-1973 cells, (NTERA-2, Nt-2; malignant pluripotent embryonal carcinoma), Pancreatic β-cells, Prenatal cardiomyocytes (hCM), Primary dendritic cells, Primary fibroblasts (hFB), Primary foreskin fibroblasts (HFF), Primary hepatic stellate cells, Primary hepatocytes, Primary neural cells, Primary umbilical vein endothelial cell (HUVEC), Saos-2 cells, SH-SY5Y cells, SK-BR-3 cells, SK-N-MC cells, U-2 OS, Umbilical cord blood stem cells (uMSC), W12 cells, WI38 cells, Non-human primate cells, COS-7 cells, CV-1 cells, Vero cells, Rodent cells, Hamster cells, CHO cells, CHO KI cells, CHO M1WT3 cells, Potoroo (Rat Kangaroo) cells, Ptk2 cells, BHK cells, RGM I cells, PC12 cells, Primary rat chondrocytes, Rat2 cells, Mouse cells, L929 cells, Mouse pancreatic β-cells, Mouse primary kidney cells, N2a cells, NIH 3T3 cells, Primary rat hepatocyts, Rat Brain Pericytes, Porcine cells, CPK cells (porcine kindney), FS-L3 cells, PK-15 cells, adult porcine stem cells, Porcine coronary artery smooth muscle cells (pCSMC), LLC-PK1 cells, Primary Cardiac Smooth Muscle Cells, Bovine cells, MDB cells, BT cells, Ovine cells, FLL-YFT cells, Deer cells, Indian Muntjac cells, Fox Cells, FoLu cells, Canine Cells, MDCK (NBL-2) cells, Chicken primary myoblasts, Chicken whole embryonic fibroblast cells, OMK cells, EPC cells, CHH-1 cells or a human cell. The cell may be a primary cell or it may be a cell of an established cell line. If desired, the virus may be introduced into a primary cell culture approximately 24 hours after plating of the primary cell culture to maximize the efficiency of infection. Preferably, the mammalian cell is a hepatocyte, such as a HepG2 cell or a primary hepatocyte; a cell of the kidney cell line 293; or a PC12 cell (e.g., a differentiated PC12 cell induced by nerve growth factor). Other suitable mammalian cells are those which have an asialoglycoprotein receptor. Additional suitable mammalian cells include NIH3T3 cells, HeLa cells, Cos 7 cells, CHO cells and C₂-C₁₂ cells.

The virus can be introduced into the cell in vitro, or in vivo. Where the virus is introduced into a cell in vitro, the cell can subsequently be introduced into a mammal (e.g., into the portal vein or into the spleen), if desired.

If desired, the virus may be introduced into the cell by administering the virus to a mammal which carries the cell. For example, the virus can be administered intravenously or intraperitoneally to such a mammal. If desired, a slow-release device, such as an implantable pump, may be used to facilitate delivery of the virus to a cell. Where the virus is administered to a mammal carrying the cell into which the virus will be introduced, the cell can be targeted by modulating the amount of the virus administered to the mammal and by controlling the method of delivery. For example, intravascular administration of the virus to the portal vein or to the hepatic artery may be used to facilitate targeting the virus to a liver cell. In another method, the virus may be administered to a cell or organ of a donor individual prior to transplantation of the cell or organ to a recipient.

Certain embodiments of the invention include contacting a mammalian cell with a recombinant virus comprising a genome of a non-mammalian DNA virus that includes a mammalian promoter, and a fluorescent protein coding sequence operably linked to the mammalian promoter. The DNA virus can optionally include all genes necessary for the virus to replicate in its natural host, or can have 1 or more of such genes inactivated or deleted. To carry out this method for example, general cell culture and viral infection methods can be used (See e.g., Boyce and Bucher (Baculovirus-mediated gene transfer into mammalian cells): Proc. Natl. Acad. Sci. USA: 93:2348 (1996), incorporated by reference in its entirety; or Premo™ Cameleon Calcium Sensor product manual (Invitrogen Corporation, Carlsbad, Calif.)). Where the cell is allowed to live under in vitro conditions, conventional tissue culture conditions and methods may be used. Briefly, a tissue culture vessel can be inoculated and cells allowed to grow, and optionally attach, depending on the cell type. The cell can be allowed to grow, for example for 1 hour to 2 days, 2 hours to 1.5 days, or 4 hours to 1 day. Then medium can be aspirated and a recombinant virus of the invention, for example diluted in a buffer such as PBS, can be applied to the cells for 15 minutes to 72 hours, or in an illustrative embodiment for 2-4 hours, or for 5-60 minutes, or for 15-30 minutes for stem cell or primary cell cultures. After the incubation with virus, the viral infection media can then be replaced with growth media that can include an enhancer, as disclosed herein, for 15 minutes to 8 hours, or from 1-4 hours, or from 1.5-2 hours at 37 C. Cells can then be grown in media and analyzed. In some embodiments, the cell is allowed to live on a substrate which contains collagen, such as Type I collagen or rat tail collagen, or a matrix containing laminin. Implantable versions of such substrates are also suitable for use in the invention (see, e.g., Hubbell et al., 1995, Bio/Technology 13:565-576 and Langer and Vacanti, 1993, Science 260: 920-925). As an alternative to, or in addition to, allowing the cell to live under in vitro conditions, the cell can be allowed to live under in vivo conditions (e.g., in a human).

If desired, the virus genome can be engineered to express more than one exogenous gene (e.g., the virus can be engineered to express OTC and AS). Accordingly, Another embodiment provided herein, is a method for expressing two or more exogenous genes in a mammalian cell comprising contacting a non-mammalian DNA virus with the mammalian cell such that the non-mammalian DNA virus infects the mammalian cell and the exogenous genes are expressed, wherein the two or more exogenous genes encode a genetically-encoded sensor and a target protein. The target protein, in illustrative embodiments is a second messenger system target protein. For example, in this embodiment, the non-mammalian DNA virus can encode an ion channel protein and a second messenger sensor, such as a calcium sensor. In a related embodiment, the present invention provides a non-mammalian DNA virus that encodes two or more exogenous genes each operably linked to two or more mammalian promoters, wherein the two or more genes encode a genetically-encoded sensor and a second messenger system target protein. A method and virus of this embodiment of the invention can be used, for example, in drug-discovery experiments where a cell expressing the two or more exogenous genes encoded by the non-mammalian DNA virus are contacted with a test molecule, such as a small-organic molecule or other test compound. The test compound can be a member of a population of test compounds produced by combinatorial chemistry, for example.

In these embodiments that include expressing two or more exogenous genes, the second messenger target protein can be, as non-limiting but illustrative examples, a GPCR (G-protein coupled receptor), a kinase, a nuclear receptor, an ion channel, a G-protein, a transporter, a transcription factor, a glycosidase/glycosyltransferase, a phosphodiesterases, a proteases, and a protein phosphatases. The genetically-encoded sensor can be a second messenger sensor such as, but not limited to, a calcium sensor, such as a Cameleon sensor, a cAMP sensor, an IP3 sensor, a redox sensor, a pH sensor, or an AKAR kinase sensor. The sensor, typically includes a fluorescent or luminescent protein, such as BFP, CFP, GFP, YFP, OFP, or RFP. Either the second messenger target protein or the sensor protein can be fusion protein that include an intracellular targeting sequence.

The first exogensous gene can be operably linked to a first mammalian promoter and the second exogenous gene can be operably linked to the first promoter or to a second mammalian promoter.

In certain illustrative examples of these embodiments that include expressing two or more exogenous genes, the cell can be a cell that does not naturally express the second messenger system protein, or expresses the second messenger system protein at a relatively low level compared to the expression level from the non-mammalian DNA virus.

Embodiments of the invention are interchangeable. Accordingly a teaching in one section of the specification should not be limited to that section unless the relevant section of the specification explicitly states this. It will be understood that although the specification explicitly discloses embodiments that include a non-mammalian DNA virus, a mammalian promoter, and optionally a mammalian cell, the invention includes embodiments that include a virus having a natural host of a first species and a promoter from a second species, which is different from the first species. In certain aspects the first species is a non-mammalian species, for example a non-mammalian eukaryotic species, such as, but not limited to, an insect species, a protozoan species, a plant species, or a fish species. In certain non-limiting illustrative embodiments wherein the first species is a non-mammalian sepecies, the second species can be a mammalian species, such as a human species. Not to be limited by theory, an advantage to this system that utilizes a virus that infests a first host cell species and a promoter from a second species, is that when used as a research product, the product is not as conveniently replicated by a laboratory using the DNA virus in an assay.

Accordingly, provided herein is a virus having a first species as its natural host, wherein the virus encodes two or more exogenous genes operably linked to one or more promoters of a second species, wherein the two or more genes encode a genetically-encoded sensor and a second messenger system protein. For example, the DNA virus can encode a genetically encoded sensor operably linked to a first promoter, and a second messenger system protein operably linked to the first promoter or to a second promoter.

Also provided is a method for selling a fluorescent sensor, reporter molecule, and/or kit provided herein. The method can include, for example, presenting to a customer an identifier that identifies the fluorescent sensor, reporter molecule, and/or kit provided herein, and providing access to the customer to a purchase function for purchasing the fluorescent sensor, reporter molecule, and/or kit provided herein using the identifier. The identifier is typically presented to the customer as part of an ordering system. The ordering system can include an input function for identifying a desired product, and a purchasing function for purchasing a desired product that is identified. The ordering system is typically under the direct or indirect control of a provider. A customer as used herein, refers to any individual, institution, corporation, university, or organization seeking to obtain biological research products and services. A provider as used herein, refers to any individual, institution, corporation, university, or organization seeking to provide biological research products and services. The ordering system can include a computer program stored in a computer storage device such as a hard drive.

The present invention also provides a method for selling a viral particle, composition, fluorescent sensor, reporter molecule, and/or kit provided herein, comprising: presenting to a customer an input function of a telephonic ordering system, and/or presenting to a customer a data entry field or selectable list of entries as part of a computer system, wherein the fluorescent sensor, reporter molecule, or kit is identified using the input function. Where the input function is part of a computer system, such as displayed on one or more pages of an Internet site, the customer is typically presented with an on-line purchasing function, such as an online shopping cart, wherein the purchasing function is used by the customer to purchase the identified fluorescent sensor, reporter molecule, and/or kit. In one aspect, a plurality of identifiers are provided to a customer, each identifying a different fluorescent sensor, reporter molecule, and/or kit provided herein, or a different volume, weight, or size of the fluorescent sensor, reporter molecule, and/or kit provided herein. The method may further comprise activating the purchasing function to purchase the fluorescent sensor, reporter molecule, and/or kit provided herein. The method may still further comprise shipping the purchased fluorescent sensor, reporter molecule, and/or kit provided herein to the customer. The fluorescent sensor, reporter molecule, and/or kit can be shipped by a provider to the customer. The provider typically controls the input function, and can control the web site accessed to access the input function to purchase a sensor, reporter molecule, and/or kit provided herein.

A kit as provided herein, typically comprises a viral particle comprising a genome of a non-mammalian DNA virus and an exogenous reporter gene operably linked to a mammalian promoter. In certain embodiments, a gene required for replication is deleted or inactivated. The kit can further comprise one or more reagents used for the detection of the fluorescent sensor. For example, the kit can include one or more developers and/or enhancers and one or more solvents for the developer or enhancer. For example, the kit can include an enhancer, such as a de-acetylase inhibitor, for example Trichostatin A. The kit can also include a vial of DMSO for reconstituting the enhancer, such as for reconstituting the Trichostatin A.

In another aspect, provided herein is a method for providing a mammalian cell reporter vector, that includes offering the mammalian cell reporter vector for sale to a customer along with the right to use the mammalian cell reporter vector but not the right to replicate the mammalian cell reporter vector. In illustrative embodiments, the mammalian cell reporter vector is a non-mammalian DNA virus that includes a mammalian promoter or a mammalian-active promoter, operatively linked to a reporter gene, such as a fluorescent protein.

In certain aspects, the method or kit can include a series of cell reporter vectors, such as a series of mammalian cell reporter vectors, that each encode a different reporter. The reporters can emit different colors. For example, the reporters can be fluorescent proteins that emit light at different wavelengths, such as the wavelengths of different colors such as red, green, blue, yellow, cyan, orange, etc. In these aspects, the cell reporter vectors can include all the genes necessary for replication in a host cell, such as a mammalian or non-mammalian host cell, or can be inactivated with respect to one or more of such genes. The series of cell reporter vectors can include 2, 3, 4, 5, 6, 7, 8, 9, 10, 11, 12, 13, 14, 15, 20, or 25 vectors each comprising a different reporter, such as different fluorescent protein, and/or each comprising a different combination of targeting sequence and reporter, such as a different fluorescent protein.

In certain illustrative examples of kits and methods provided herein, the kits include or are associated with a limited use label license that prohibits replication of the DNA virus and/or permits use of the DNA virus to infect mammalian cells. In other aspects, the limited use label license can prohibit use of the virus in cells in which it is capable of replicating, or limit use rights to only cells in which it is not replicable. For example, where the viral particle in a kit is capable of infecting insect cells but not mammalian cells, the limited use label license in an illustrative embodiments permits use only in mammalian cells, or in cells that include mammalian cells but not insect cells. These aspects with limited use label licenses that prohibit replication or infection of certain cell types or that limit use to certain cell types, in illustrative aspects, are included with kits or associated with methods in which the reporter vector included therein retains the ability to replicate in one or more host cells because they have not been engineered to not retain such ability. The limited use label licenses can be included in a kit or manual or associated therewith for example, by electronic association on an Internet site.

### EXAMPLES

### Example 1

### Transduction of Cells with a Baculovirus Expressing a Targeted Fluorescence Protein

A non-limiting example of one embodiment of the invention is called ORGANELLE LIGHTS™ reagents (intent to use trademark of Invitrogen Corporation (Carlsbad, Calif.)). ORGANELLE LIGHTS™ are a fusion protein between a targeting sequence and a fluorescent protein which is expressed by a baculovirus. Specific examples along with the DNA sequence of the targeting sequence-fluorescent protein fusion protein follow.

Organelle Lights™ Nuc-GFP

SEQ ID:11

CACCATGGTGAGCAAGGGCGAGGAGCTGTTCACCGGGGTGGTGCCCATCC
TGGTCGAGCTGGACGGCGACGTAAACGGCCACAAGTTCAGCGTGTCCGGC
GAGGGCGAGGGCGATGCCACCTACGGCAAGCTGACCCTGAAGTTCATCTG
CACCACCGGCAAGCTGCCCGTGCCCTGGCCCACCCTCGTGACCACCTTCA
CCTACGGCGTGCAGTGCTTCGCCCGCTACCCCGACCACATGAAGCAGCAC
GACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCG
AGGGCGACACCCTGGTGAACCGCATCGAGCTGAAGGGCATCGACTTCAAG
GAGGACGGCAACATCCTGGGGCACAAGCTGGAGTACAACTACAACAGCCA
CAAGGTCTATATCACCGCCGACAAGCAGAAGAACGGCATCAAGGTGAACT
TCAAGACCCGCCACAACATCGAGGACGGCAGCGTGCAGCTCGCCGACCAC
TACCAGCAGAACACCCCCATCGGCGACGGCCCCGTGCTGCTGCCCGACAA
CCACTACCTGAGCACCCAGTCCGCCCTGAGCAAAGACCCCAACGAGAAGC
GCGATCACATGGTCCTGCTGGAGTTCGTGACCGCCGCCGGGAT

Organelle Lights™ Mito-GFP

SEQ ID: 12

CACCATGCGCAAGATGCTGGCCGCCGTGTCCCGTGTGCTCTCCGGCGCCT
CCCAGAAGCCCGCCTCCCGTGTGCTGGTGGCCTCCCGTAACGTGAGCAAG
GGCGAGGAGCTGTTCACCGGGGTGGTGCCCATCCTGGTCGAGCTGGACGG
CGACGTAAACGGCCACAAGTTCAGCGTGTCCGGCGAGGGCGAGGGCGATG
CCACCTACGGCAAGCTGACCCTGAAGTTCATCTGCACCACCGGCAAGCTG
CCCGTGCCCTGGCCCACCCTCGTGACCACCTTCACCTACGGCGTGCAGTG
CTTCGCCCGCTACCCCGACCACATGAAGCAGCACGACTTCTTCAAGTCCG
CCATGCCCGAAGGCTACGTCCAGGAGCGCACCATCTTCTTCAAGGACGAC
GGCAACTACAAGACCCGCGCCGAGGTGAAGTTCGAGGGCGACACCCTGGT
GAACCGCATCGAGCTGAAGGGCATCGACTTCAAGGAGGACGGCAACATCC
TGGGGCACAAGCTGGAGTACAACTACAACAGCCACAAGGTCTATATCACC
GCCGACAAGCAGAAGAACGGCATCAAGGTGAACTTCAAGACCCGCCACAA
CATCGAGGACGGCAGCGTGCAGCTCGCCGACCACTACCAGCAGAACACCC
CCATCGGCGACGGCCCCGTGCTGCTGCCCGACAACCACTACCTGAGCACC
CAGTCCGCCCTGAGCAAAGACCCCAACGAGAAGCGCGATCACATGGTCCT
GCTGGAGTTCGTGACCGCCGCCGGGATCACTCTCGGCATGGACGAGCTGT
ACAAGTAAGAC

Organelle Lights™ Peroxi-GFP

SEQ ID:13

CACCATGGTGAGCAAGGGCGAGGAGCTGTTCACCGGGGTGGTGCCCATCC
TGGTCGAGCTGGACGGCGACGTAAACGGCCACAAGTTCAGCGTGTCCGGC
GAGGGCGAGGGCGATGCCACCTACGGCAAGCTGACCCTGAAGTTCATCTG
CACCACCGGCAAGCTGCCCGTGCCCTGGCCCACCCTCGTGACCACCTTCA
CCTACGGCGTGCAGTGCTTCGCCCGCTACCCCGACCACATGAAGCAGCAC
GACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCG
AGGGCGACACCCTGGTGAACCGCATCGAGCTGAAGGGCATCGACTTCAAG
GAGGACGGCAACATCCTGGGGCACAAGCTGGAGTACAACTACAACAGCCA
CAAGGTCTATATCACCGCCGACAAGCAGAAGAACGGCATCAAGGTGAACT
TCAAGACCCGCCACAACATCGAGGACGGCAGCGTGCAGCTCGCCGACCAC
TACCAGCAGAACACCCCCATCGGCGACGGCCCCGTGCTGCTGCCCGACAA
CCACTACCTGAGCACCCAGTCCGCCCTGAGCAAAGACCCCAACGAGAAGC
GCGATCACATGGTCCTGCTGGAGTTCGTGACCGCCGCCGGGATCACTCTC
GGCATGGACGAGCTGTACAAGAGCAAGCTGTAAGACCCAGCTTTCTTGTA
CAAAGTGGTCCCC

Organelle Lights™ ER-GFP

SEQ ID:14

ATGCTGCTGCCCGTGCCTCTGCTCCTGGGCCTGCTGGGCCTGGCCGCTGC
CGGAGGCGTGAGCAAGGGCGAGGAGCTGTTCACCGGGGTGGTGCCCATCC
TGGTCGAGCTGGACGGCGACGTAAACGGCCACAAGTTCAGCGTGTCCGGC
GAGGGCGAGGGCGATGCCACCTACGGCAAGCTGACCCTGAAGTTCATCTG
CACCACCGGCAAGCTGCCCGTGCCCTGGCCCACCCTCGTGACCACCTTCA
CCTACGGCGTGCAGTGCTTCGCCCGCTACCCCGACCACATGAAGCAGCAC
GACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCG
AGGGCGACACCCTGGTGAACCGCATCGAGCTGAAGGGCATCGACTTCAAG
GAGGACGGCAACATCCTGGGGCACAAGCTGGAGTACAACTACAACAGCCA
CAAGGTCTATATCACCGCCGACAAGCAGAAGAACGGCATCAAGGTGAACT
TCAAGACCCGCCACAACATCGAGGACGGCAGCGTGCAGCTCGCCGACCAC
TACCAGCAGAACACCCCCATCGGCGACGGCCCCGTGCTGCTGCCCGACAA
CCACTACCTGAGCACCCAGTCCGCCCTGAGCAAAGACCCCAACGAGAAGC
GCGATCACATGGTCCTGCTGGAGTTCGTGACCGCCGCCGGGATCACTCTC
GGCATGGACGAGCTGTACAAGAAGGACGAGCTGTAA

Organelle Lights™ NE-GFP

SEQ ID:15

CACCATGGTGAGCAAGGGCGAGGAGCTGTTCACCGGGGTGGTGCCCATCC
TGGTCGAGCTGGACGGCGACGTAAACGGCCACAAGTTCAGCGTGTCCGGC
GAGGGCGAGGGCGATGCCACCTACGGCAAGCTGACCCTGAAGTTCATCTG
CACCACCGGCAAGCTGCCCGTGCCCTGGCCCACCCTCGTGACCACCTTCA
CCTACGGCGTGCAGTGCTTCGCCCGCTACCCCGACCACATGAAGCAGCAC
GACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCG
AGGGCGACACCCTGGTGAACCGCATCGAGCTGAAGGGCATCGACTTCAAG
GAGGACGGCAACATCCTGGGGCACAAGCTGGAGTACAACTACAACAGCCA
CAAGGTCTATATCACCGCCGACAAGCAGAAGAACGGCATCAAGGTGAACT
TCAAGACCCGCCACAACATCGAGGACGGCAGCGTGCAGCTCGCCGACCAC
TACCAGCAGAACACCCCCATCGGCGACGGCCCCGTGCTGCTGCCCGACAA
CCACTACCTGAGCACCCAGTCCGCCCTGAGCAAAGACCCCAACGAGAAGC
GCGATCACATGGTCCTGCTGGAGTTCGTGACCGCCGCCGGGATCACTCTC
GGCATGGACGAGCTGTACAAGCGGGGCTTCCTGTTCCGGGTGCTGCGGGC
CGCCCTGCCCCTGCAGCTCCTGCTGCTGCTCCTGATCGGCCTGGCCTGCC
TGGTGCCCATGAGCGAGGAGGACTACAGCTGCGCCCTGAGCAACAACTTC
GCCCGGAGCTTCCATCCCATGCTGCGGTACACCAACGGCCCTCCACCCCT
GTAAGAC

Organelle Lights™ PM-GFP

SEQ ID:16

CACCATGGGCTGCGTGTGCTCCCGTGGCGGCGTGAGCAAGGGCGAGGAGC
TGTTCACCGGGGTGGTGCCCATCCTGGTCGAGCTGGACGGCGACGTAAAC
GGCCACAAGTTCAGCGTGTCCGGCGAGGGCGAGGGCGATGCCACCTACGG
CAAGCTGACCCTGAAGTTCATCTGCACCACCGGCAAGCTGCCCGTGCCCT
GGCCCACCCTCGTGACCACCTTCACCTACGGCGTGCAGTGCTTCGCCCGC
TACCCCGACCACATGAAGCAGCACGACTTCTTCAAGTCCGCCATGCCCGA
AGGCTACGTCCAGGAGCGCACCATCTTCTTCAAGGACGACGGCAACTACA
AGACCCGCGCCGAGGTGAAGTTCGAGGGCGACACCCTGGTGAACCGCATC
GAGCTGAAGGGCATCGACTTCAAGGAGGACGGCAACATCCTGGGGCACAA
GCTGGAGTACAACTACAACAGCCACAAGGTCTATATCACCGCCGACAAGC
AGAAGAACGGCATCAAGGTGAACTTCAAGACCCGCCACAACATCGAGGAC
GGCAGCGTGCAGCTCGCCGACCACTACCAGCAGAACACCCCCATCGGCGA
CGGCCCCGTGCTGCTGCCCGACAACCACTACCTGAGCACCCAGTCCGCCC
TGAGCAAAGACCCCAACGAGAAGCGCGATCACATGGTCCTGCTGGAGTTC
GTGACCGCCGCCGGGATCACTCTCGGCATGGACGAGCTGTACAAGTAAGA
CCCAGCTTTCTTGTACAAAGTGGTCCCC

Organelle Lights™ Golgi-GFP

SEQ ID:17

CACCATGCGGCGGCGCTCGCGGATGCTGCTCTGCTTCGCCTTCCTGTGGG
TGCTGGGCATCGCCTACTACATGTACTCGGGGGGCGGATCTGCGCTGGCC
GGAGGCGCTGGCGGAGGCGCCGGCAGGAAGGAGGACTGGAATGAAATTGA
CCCCATTAAAAAGAAAGACCTTCATCACAGCAATGGAGAAGAGAAAGCAC
AAAGCATGGAGACCCTCCCTCCAGGGAAAGTACGGTGGCCAGACTTTAAC
CAGGAAGCTTATGTTGGAGGGACGATGGTCCGCTCCGGGCAGGACCCTTA
CGCCCGCAACAAGTTCAACCAGGTGGAGAGTGATAAGCTTCGGGCGGGAG
CCGTGAGCAAGGGCGAGGAGCTGTTCACCGGGGTGGTGCCCATCCTGGTC
GAGCTGGACGGCGACGTAAACGGCCACAAGTTCAGCGTGTCCGGCGAGGG
CGAGGGCGATGCCACCTACGGCAAGCTGACCCTGAAGTTCATCTGCACCA
CCGGCAAGCTGCCCGTGCCCTGGCCCACCCTCGTGACCACCTTCACCTAC
GGCGTGCAGTGCTTCGCCCGCTACCCCGACCACATGAAGCAGCACGACTT
CTTCAAGTCCGCCATGCCCGAAGGCTACGTCCAGGAGCGCACCATCTTCT
TCAAGGACGACGGCAACTACAAGACCCGCGCCGAGGTGAAGTTCGAGGGC
GACACCCTGGTGAACCGCATCGAGCTGAAGGGCATCGACTTCAAGGAGGA
CGGCAACATCCTGGGGCACAAGCTGGAGTACAACTACAACAGCCACAAGG
TCTATATCACCGCCGACAAGCAGAAGAACGGCATCAAGGTGAACTTCAAG
ACCCGCCACAACATCGAGGACGGCAGCGTGCAGCTCGCCGACCACTACCA
GCAGAACACCCCCATCGGCGACGGCCCCGTGCTGCTGCCCGACAACCACT
ACCTGAGCACCCAGTCCGCCCTGAGCAAAGACCCCAACGAGAAGCGCGAT
CACATGGTCCTGCTGGAGTTCGTGACCGCCGCCGGGATCACTCTCGGCAT
GGACGAGCTGTACAAGTAAGACC

Organelle Lights™ PM-CFP

SEQ ID:18

CACCATGGGCTGCGTGTGCTCCCGTGGCGGCGTGAGCAAGGGCGAGGAGC
TGTTCACCGGGGTGGTGCCCATCCTGGTCGAGCTGGACGGCGACGTAAAC
GGCCACAGGTTCAGCGTGTCCGGCGAGGGCGAGGGCGATGCCACCTACGG
CAAGCTGACCCTGAAATTCATCTGCACCACCGGCAAGCTGCCCGTGCCCT
GGCCCACCCTCGTGACCACCTTCGCCTGGGGCGTGCAGTGCTTCGCCCGC
TACCCCGACCACATGAAGCAGCACGACTTCTTCAAGTCCGCCATGCCCGA
AGGCTACGTCCAGGAGCGTACCATCTTCTTCAAGGACGACGGCAACTACA
AGACCCGCGCCGAGGTGAAGTTCGAGGGCGACACCCTGGTGAACCGCATC
GAGCTGAAGGGCATCGACTTCAAGGAGGACGGCAACATCCTGGGGCACAA
GCTGGAGTACAACTACATCAGCCACAACGTCTATATCACCGCCGACAAGC
AGAAGAACGGCATCAAGGCCCACTTCAAGATCCGCCACAACATCGAGGAC
GGCAGCGTGCAGCTCGCCGACCACTACCAGCAGAACACCCCCATCGGCGA
CGGCCCCGTGCTGCTGCCCGACAACCACTACCTGAGCACCCAGTCCGCCC
TGAGCAAAGACCCCAACGAGAAGCGCGATCACATGGTCCTGCTGGAGTTC
GTGACCGCCGCCGGGATCACTCTCGGCATGGACGAGCTGTACAAGTAA

Organelle Lights™ PM-YFP

SEQ ID:19

CACCATGGGCTGCGTGTGCTCCCGTGGCGGCGTGAGCAAGGGCGAGGAGC
TGTTCACCGGGGTGGTGCCCATCCTGGTCGAGCTGGACGGCGACGTAAAC
GGCCACAAGTTCAGCGTGTCCGGCGAGGGCGAGGGCGATGCCACCTACGG
CAAGCTGACCCTGAAGTTCATCTGCACCACCGGCAAGCTGCCCGTGCCCT
GGCCCACCCTCGTGACCACCTTCGGCTACGGCGTGCAGTGCTTCGCCCGC
TACCCCGACCACATGCGCCAGCACGACTTCTTCAAGTCCGCCATGCCCGA
AGGCTACGTCCAGGAGCGCACCATCTTCTTCAAGGACGACGGCAACTACA
AGACCCGCGCCGAGGTGAAGTTCGAGGGCGACACCCTGGTGAACCGCATC
GAGCTGAAGGGCATCGACTTCAAGGAGGACGGCAACATCCTGGGGCACAA
GCTGGAGTACAACTACAACAGCCACAACGTCTATATCATGGCCGACAAGC
AGAAGAACGGCATCAAGGTGAACTTCAAGATCCGCCACAACATCGAGGAC
GGCAGCGTGCAGCTCGCCGACCACTACCAGCAGAACACCCCCATCGGCGA
CGGCCCCGTGCTGCTGCCCGACAACCACTACCTGAGCTACCAGTCCGCCC
TGAGCAAAGACCCCAACGAGAAGCGCGATCACATGGTCCTGCTGGAGTTC
GTGACCGCCGCCGGGATCACTCTCGGCATGGACGAGCTGTACAAGTAA

Organelle Lights™ Nuc-CFP

SEQ ID:20

CACCATGGTGAGCAAGGGCGAGGAGCTGTTCACCGGGGTGGTGCCCATCC
TGGTCGAGCTGGACGGCGACGTAAACGGCCACAGGTTCAGCGTGTCCGGC
GAGGGCGAGGGCGATGCCACCTACGGCAAGCTGACCCTGAAGTTCATCTG
CACCACCGGCAAGCTGCCCGTGCCCTGGCCCACCCTCGTGACCACCTTCG
CCTGGGGCGTGCAGTGCTTCGCCCGCTACCCCGACCACATGAAGCAGCAC
GACTTCTTCAAGTCCGCCATGCCCGAAGGCTACGTCCAGGAGCGTACCAT
CTTCTTCAAGGACGACGGCAACTACAAGACCCGCGCCGAGGTGAAGTTCG
AGGGCGACACCCTGGTGAACCGCATCGAGCTGAAGGGCATCGACTTCAAG
GAGGACGGCAACATCCTGGGGCACAAGCTGGAGTACAACTACATCAGCCA
CAACGTCTATATCACCGCCGACAAGCAGAAGAACGGCATCAAGGCCCACT
TCAAGATCCGCCACAACATCGAGGACGGCAGCGTGCAGCTCGCCGACCAC
TACCAGCAGAACACCCCCATCGGCGACGGCCCCGTGCTGCTGCCCGACAA
CCACTACCTGAGCACCCAGTCCGCCCTGAGCAAAGACCCCAACGAGAAGC
GCGATCACATGGTCCTGCTGGAGTTCGTGACCGCCGCCGGGATCACTCTC
GGCATGGACGAGCTGTACAAGCCCAGCAAGAAGAAGCGTAAGGTGTAA

Organelle Lights™ Nuc-YFP

SEQ ID:21

CACCATGGTGAGCAAGGGCGAGGAGCTGTTCACCGGGGTGGTGCCCATCC
TGGTCGAGCTGGACGGCGACGTAAACGGCCACAAGTTCAGCGTGTCCGGC
GAGGGCGAGGGCGATGCCACCTACGGCAAGCTGACCCTGAAGTTCATCTG
CACCACCGGCAAGCTGCCCGTGCCCTGGCCCACCCTCGTGACCACCTTCG
GCTACGGCGTGCAGTGCTTCGCCCGCTACCCCGACCACATGCGCCAGCAC
GACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCG
AGGGCGACACCCTGGTGAACCGCATCGAGCTGAAGGGCATCGACTTCAAG
GAGGACGGCAACATCCTGGGGCACAAGCTGGAGTACAACTACAACAGCCA
CAACGTCTATATCATGGCCGACAAGCAGAAGAACGGCATCAAGGTGAACT
TCAAGATCCGCCACAACATCGAGGACGGCAGCGTGCAGCTCGCCGACCAC
TACCAGCAGAACACCCCCATCGGCGACGGCCCCGTGCTGCTGCCCGACAA
CCACTACCTGAGCTACCAGTCCGCCCTGAGCAAAGACCCCAACGAGAAGC
GCGATCACATGGTCCTGCTGGAGTTCGTGACCGCCGCCGGGATCACTCTC
GGCATGGACGAGCTGTACAAGCCCAGCAAGAAGAAGCGTAAGGTGTAA

Organelle Lights™ Nuc-OFP

SEQ ID:22

CACCGAGCTCATGAACCTGAGCAAAAACGTGAGCGTGAGCGTGTATATGA
AGGGGAACGTCAACAATCATGAGTTTGAGTACGACGGGGAAGGTGGTGGT
GATCCTTATACAGGTAAATATTCCATGAAGATGACGCTACGTGGTCAAAA
TTCCCTACCCTTTTCCTATGATATCATTACCACGGCATTTCAGTATGGTT
TCCGCGTATTTACAAAATACCCTGAGGGAATTGTTGACTATTTTAAGGAT
TCGCTTCCCGACGCATTCCAGTGGAACAGACGAATTGTGTTTGAAGATGG
TGGAGTACTAAACATGAGCAGTGATATCACATATAAAGATAATGTTCTGC
ATGGTGACGTCAAAGCTGAAGGAGTGAACTTCCCGCCGAATGGGCCAGTG
ATGAAGAATGAAATTGTGATGGAGGAACCGACTGAAGAAACATTTACTCC
AAAAAACGGGGTTCTTGTTGGCTTTTGTCCCAAAGCGTACTTACTTAAAG
ATGGTTCCTATTACTATGGAAATATGACAACATTTTACAGATCCAAGAAA
TCTGGCCAGGCACCTCCTGGGTATCACTTTGTTAAGCATCGTCTCGTCAA
GACCAATGTGGGACATGGATTTAAGACGGTTGAGCAGACTGAATATGCCA
CTGCTCATGTCAGTGATCTTCCCAAGCTTCCCAGCAAGAAGAAGCGTAAG
GTGTAAGAC

Organelle Lights™ NE-OFP,

SEQ ID:23

ATGGAGCTCAACCTGAGCAAAAACGTGAGCGTGAGCGTGTATATGAAGGG
GAACGTCAACAATCATGAGTTTGAGTACGACGGGGAAGGTGGTGGTGATC
CTTATACAGGTAAATATTCCATGAAGATGACGCTACGTGGTCAAAATTCC
CTACCCTTTTCCTATGATATCATTACCACGGCATTTCAGTATGGTTTCCG
CGTATTTACAAAATACCCTGAGGGAATTGTTGACTATTTTAAGGATTCGC
TTCCCGACGCATTCCAGTGGAACAGACGAATTGTGTTTGAAGATGGTGGA
GTACTAAACATGAGCAGTGATATCACATATAAAGATAATGTTCTGCATGG
TGACGTCAAGGCTGAGGGAGTGAACTTCCCGCCGAATGGGCCAGTGATGA
AGAATGAAATTGTGATGGAGGAACCGACTGAAGAAACATTTACTCCAAAA
AACGGGGTTCTTGTTGGCTTTTGTCCCAAAGCGTACTTACTTAAAGATGG
TTCCTATTACTATGGAAATATGACAACATTTTACAGATCCAAGAAATCTG
GCCAGGCACCTCCTGGGTATCACTTTGTTAAGCATCGTCTCGTCAAGACC
AATGTGGGACATGGATTTAAGACGGTTGAGCAGACTGAATATGCCACTGC
TCATGTCAGTGATCTTCCCAAGAAGCTTCGGGGCTTCCTGTTCCGGGTGC
TGCGGGCCGCCCTGCCCCTGCAGCTCCTGCTGCTGCTCCTGATCGGCCTG
GCCTGCCTGGTGCCCATGAGCGAGGAGGACTACAGCTGCGCCCTGAGCAA
CAACTTCGCCCGGAGCTTCCATCCCATGCTGCGGTACACCAACGGCCCTC
CACCCCTGTGA

Organelle Lights™ Mito-OFP

SEQ ID:24

ATGCGCAAGATGCTGGCCGCCGTGTCCCGTGTGCTCTCCGGCGCCTCCCA
GAAGCCCGCCTCCCGTGTGCTGGTGGCCTCCCGTAACGAGCTCAACCTGA
GCAAAAACGTGAGCGTGAGCGTGTATATGAAGGGGAACGTCAACAATCAT
GAGTTTGAGTACGACGGGGAAGGTGGTGGTGATCCTTATACAGGTAAATA
TTCCATGAAGATGACGCTACGTGGTCAAAATTCCCTACCCTTTTCCTATG
ATATCATTACCACGGCATTTCAGTATGGTTTCCGCGTATTTACAAAATAC
CCTGAGGGAATTGTTGACTATTTTAAGGATTCGCTTCCCGACGCATTCCA
GTGGAACAGACGAATTGTGTTTGAAGATGGTGGAGTACTAAACATGAGCA
GTGATATCACATATAAAGATAATGTTCTGCATGGTGACGTCAAGGCTGAG
GGAGTGAACTTCCCGCCGAATGGGCCAGTGATGAAGAATGAAATTGTGAT
GGAGGAACCGACTGAAGAAACATTTACTCCAAAAAACGGGGTTCTTGTTG
GCTTTTGTCCCAAAGCGTACTTACTTAAAGATGGTTCCTATTACTATGGA
AATATGACAACATTTTACAGATCCAAGAAATCTGGCCAGGCACCTCCTGG
GTATCACTTTGTTAAGCATCGTCTCGTCAAGACCAATGTGGGACATGGAT
TTAAGACGGTTGAGCAGACTGAATATGCCACTGCTCATGTCAGTGATCTT
CCCAAGGAAGCTTGA

Organelle Lights™ ER-OFP

SEQ ID:25

ATGCTGCTGCCCGTGCCTCTGCTCCTGGGCCTGCTGGGCCTGGCCGCTGC
CGGAGGCGAGCTCAACCTGAGCAAAAACGTGAGCGTGAGCGTGTATATGA
AGGGGAACGTCAACAATCATGAGTTTGAGTACGACGGGGAAGGTGGTGGT
GATCCTTATACAGGTAAATATTCCATGAAGATGACGCTACGTGGTCAAAA
TTCCCTACCCTTTTCCTATGATATCATTACCACGGCATTTCAGTATGGTT
TCCGCGTATTTACAAAATACCCTGAGGGAATTGTTGACTATTTTAAGGAT
TCGCTTCCCGACGCATTCCAGTGGAACAGACGAATTGTGTTTGAAGATGG
TGGAGTACTAAACATGAGCAGTGATATCACATATAAAGATAATGTTCTGC
ATGGTGACGTCAAGGCTGAGGGAGTGAACTTCCCGCCGAATGGGCCAGTG
ATGAAGAATGAAATTGTGATGGAGGAACCGACTGAAGAAACATTTACTCC
AAAAAACGGGGTTCTTGTTGGCTTTTGTCCCAAAGCGTACTTACTTAAAG
ATGGTTCCTATTACTATGGAAATATGACAACATTTTACAGATCCAAGAAA
TCTGGCCAGGCACCTCCTGGGTATCACTTTGTTAAGCATCGTCTCGTCAA
GACCAATGTGGGACATGGATTTAAGACGGTTGAGCAGACTGAATATGCCA
CTGCTCATGTCAGTGATCTTCCCAAGAAGCTTAAGGACGAGCTGTGA

Organelle Lights™ Golgi-OFP

SEQ ID:26

ATGCGGCGGCGCTCGCGGATGCTGCTCTGCTTCGCCTTCCTGTGGGTGCT
GGGCATCGCCTACTACATGTACTCGGGGGGCGGATCTGCGCTGGCCGGAG
GCGCTGGCGGAGGCGCCGGCAGGAAGGAGGACTGGAATGAAATTGACCCC
ATTAAAAAGAAAGACCTTCATCACAGCAATGGAGAAGAGAAAGCACAAAG
CATGGAGACCCTCCCTCCAGGGAAAGTACGGTGGCCAGACTTTAACCAGG
AAGCCTATGTTGGAGGGACGATGGTCCGCTCCGGGCAGGACCCTTACGCC
CGCAACAAGTTCAACCAGGTGGAGAGTGATAAGCTGCGGGCGGGAGCCGA
GCTCAACCTGAGCAAAAACGTGAGCGTGAGCGTGTATATGAAGGGGAACG
TCAACAATCATGAGTTTGAGTACGACGGGGAAGGTGGTGGTGATCCTTAT
ACAGGTAAATATTCCATGAAGATGACGCTACGTGGTCAAAATTCCCTACC
CTTTTCCTATGATATCATTACCACGGCATTTCAGTATGGTTTCCGCGTAT
TTACAAAATACCCTGAGGGAATTGTTGACTATTTTAAGGATTCGCTTCCC
GACGCATTCCAGTGGAACAGACGAATTGTGTTTGAAGATGGTGGAGTACT
AAACATGAGCAGTGATATCACATATAAAGATAATGTTCTGCATGGTGACG
TCAAGGCTGAGGGAGTGAACTTCCCGCCGAATGGGCCAGTGATGAAGAAT
GAAATTGTGATGGAGGAACCGACTGAAGAAACATTTACTCCAAAAAACGG
GGTTCTTGTTGGCTTTTGTCCCAAAGCGTACTTACTTAAAGATGGTTCCT
ATTACTATGGAAATATGACAACATTTTACAGATCCAAGAAATCTGGCCAG
GCACCTCCTGGGTATCACTTTGTTAAGCATCGTCTCGTCAAGACCAATGT
GGGACATGGATTTAAGACGGTTGAGCAGACTGAATATGCCACTGCTCATG
TCAGTGATCTTCCCAAGGAAGCTTGA

Organelle Lights™ Peroxi-OFP

SEQ ID:27

ATGAACCTGAGCAAAAACGTGAGCGTGAGCGTGTATATGAAGGGGAACGT
CAACAATCATGAGTTTGAGTACGACGGGGAAGGTGGTGGTGATCCTTATA
CAGGTAAATATTCCATGAAGATGACGCTACGTGGTCAAAATTCCCTACCC
TTTTCCTATGATATCATTACCACGGCATTTCAGTATGGTTTCCGCGTATT
TACAAAATACCCTGAGGGAATTGTTGACTATTTTAAGGATTCGCTTCCCG
ACGCATTCCAGTGGAACAGACGAATTGTGTTTGAAGATGGTGGAGTACTA
AACATGAGCAGTGATATCACATATAAAGATAATGTTCTGCATGGTGACGT
CAAAGCTGAAGGAGTGAACTTCCCGCCGAATGGGCCAGTGATGAAGAATG
AAATTGTGATGGAGGAACCGACTGAAGAAACATTTACTCCAAAAAACGGG
GTTCTTGTTGGCTTTTGTCCCAAAGCGTACTTACTTAAAGATGGTTCCTA
TTACTATGGAAATATGACAACATTTTACAGATCCAAGAAATCTGGCCAGG
CACCTCCTGGGTATCACTTTGTTAAGCATCGTCTCGTCAAGACCAATGTG
GGACATGGATTTAAGACGGTTGAGCAGACTGAATATGCCACTGCTCATGT
CAGTGATCTTCCCAAGCTTAGCAAGCTGTAA

Organelle Lights™ PM-OFP

SEQ ID:28

CACCATGGGCTGCGTGTGCTCCTGTGGCGGCGAGCTCAACCTGAGCAAAA
ACGTGAGCGTGAGCGTGTATATGAAGGGGAACGTCAACAATCATGAGTTT
GAGTACGACGGGGAAGGTGGTGGTGATCCTTATACAGGTAAATATTCCAT
GAAGATGACGCTACGTGGTCAAAATTCCCTACCCTTTTCCTATGATATCA
TTACCACGGCATTTCAGTATGGTTTCCGCGTATTTACAAAATACCCTGAG
GGAATTGTTGACTATTTTAAGGATTCGCTTCCCGACGCATTCCAGTGGAA
CAGACGAATTGTGTTTGAAGATGGTGGAGTACTAAACATGAGCAGTGATA
TCACATATAAAGATAATGTTCTGCATGGTGACGTCAAAGCTGAAGGAGTG
AACTTCCCGCCGAATGGGCCAGTGATGAAGAATGAAATTGTGATGGAGGA
ACCGACTGAAGAAACATTTACTCCAAAAAACGGGGTTCTTGTTGGCTTTT
GTCCCAAAGCGTACTTACTTAAAGATGGTTCCTATTACTATGGAAATATG
ACAACATTTTACAGATCCAAGAAATCTGGCCAGGCACCTCCTGGGTATCA
CTTTGTTAAGCATCGTCTCGTCAAGACCAATGTGGGACATGGATTTAAGA
CGGTTGAGCAGACTGAATATGCCACTGCTCATGTCAGTGATCTTCCCAAG
TTCGAAGCTTGAGAC

Organelle Lights™ Cyto-GFP

SEQ ID:29

CACCATGGTGAGCAAGGGCGAGGAGCTGTTCACCGGGGTGGTGCCCATCC
TGGTCGAGCTGGACGGCGACGTAAACGGCCACAAGTTCAGCGTGTCCGGC
GAGGGCGAGGGCGATGCCACCTACGGCAAGCTGACCCTGAAGTTCATCTG
CACCAGCGGCAAGCTGCCCGTGCCCTGGCCCACCCTCGTGACCACCTTCA
CCTACGGCGTGCAGTGCTTCGCCCGCTACCCCGACCACATGAAGCAGCAC
GACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCG
AGGGCGACACCCTGGTGAACCGCATCGAGCTGAAGGGCATCGACTTCAAG
GAGGACGGCAACATCCTGGGGCACAAGCTGGAGTACAACTACAACAGCCA
CAAGGTCTATATCACCGCCGACAAGCAGAAGAACGGCATCAAGGTGAACT
TCAAGACCCGCCACAACATCGAGGACGGCAGCGTGCAGCTCGCCGACCAC
TACCAGCAGAACACCCCCATCGGCGACGGCCCCGTGCTGCTGCCCGACAA
CCACTACCTGAGCACCCAGTCCGCCCTGAGCAAAGACCCCAACGAGAAGC
GCGATCACATGGTCCTGCTGGAGTTCGTGACCGCCGCCGGGATCACTCTC
GGCATGGACGAGCTGTACAAGAAGCGGCTGGAGGAGCTGCTGTACAAGAT
GTTCCTGCACACCTAAGAC

The cells to be transduced are plated at ˜1×10⁶ cells/well in a 100 mm dish and allowed to adhere and grow for approximately 4-24 hours at 37° C. and 5% CO₂ before proceeding with the transduction. An accurate cell count is important.

The entire vial of Enhancer (Component B, Trichostatin A, a de-acetylase inhibitor) should be reconstituted in 25 μL DMSO (Component C). The Enhancer solution is stable to multiple freeze/thaw cycles following the recommended storage conditions. If desired, the solution can be aliquoted following reconstitution to minimize the number of freeze/thaw cycles.

If desired, initial transduction experiments with a particular cell type can explore a range of virus concentrations around the dilution shown below to determine the optimal amount of virus to use. For all colors of these fluorescent proteins, too little virus will result in effectively transduction in only a few cells. In the OFP viruses, too much virus may result in aggregation and mistargeting as OFP is an obligate dimer.

Prepare 5.5 mL of Organelle Lights transduction solution in Dulbecco's Phosphate Buffered Saline (DPBS) without Ca++/Mg++(DPBS) (Gibco™ Cat. No. 14190) by combining 2.0 mL Organelle Lights transduction reagent (Component A) with 3.5 mL DPBS. The Organelle Lights™ transduction reagent (component A) should always be protected from light and placed back at 4° C. as soon as possible after each use. Exposure to light over time will decrease the viral titer.

Aspirate cell culture media from adherent cells in a 100 mm dish. Then add 5.5 mL of the diluted Organelle Lights transduction solution to the plate containing ˜1×10⁶ cells. Incubate the cells at room temperature (20-25° C.) in the dark for 2-4 hr with gentle rocking or shaking. Some cell types (i.e. Primary and Stem cells) are sensitive to lack of calcium and magnesium and will begin to detach. Shorter incubation times (15-30 min) can be used with these cell types.

Aspirate Organelle Lights transduction solution from the cell culture dish.

Add appropriate cell culture media with or without serum plus 1× Enhancer (i.e. 10 μL of Enhancer per 10 mL media). Incubate cells for 2 hours in optimal growth conditions (i.e. 37° C. and 5% CO₂).

Aspirate enhancer solution from the cell culture dish and add back the appropriate cell culture media. Incubate cells at 37° C. and 5% CO₂ for >16 hours to allow for expression of the Organelle Lights™.

Cells are now ready to be imaged. Imaging can be performed on either live cells or fixed cells.

Cells expressing Organelle Lights™ should be imaged using a fluorescence microscope or other suitable fluorescence imaging instrument. Approximate fluorescence excitation and emission wavelengths in nanometers are: 435/485 for CFP; 485/520 for GFP, 500/535 for YFP, (CFP, GFP, and YFP can all be seen through a FITC filter) 550/580 for OFP (Cy3™ or TRITC filters are acceptable). Fluorescence from Organelle Lights™ can be seen 16-24 hours following transduction and generally persists for 5-7 or more days. Some variation in expression level from cell to cell within the same culture is normal.

Optionally, Cells may be fixed with paraformaldehyde. Organelle Lights™ fluorescence is well-retained after paraformaldehyde fixation. To fix cells treat with 4% paraformaldehyde solution in PBS for 10-30 minutes at room temperature. If desired, cells can be permeabilized following fixation with 0.2% Triton® X-100 in PBS for 5 minutes at room temperature. The use of methanol should be avoided as it may cause loss of fluorescence.

Following fixation cells should be washed by performing two sequential five minute washes in PBS. Finally cells can be placed in PBS and imaged, or for increased photostability place cells in ProLong® Gold antifade reagent (P36930) for imaging.

What is claimed is:

1. A recombinant virus comprising a genome of a non-mammalian DNA virus optionally including the genes necessary for the virus to replicate in its natural host, a mammalian promoter, and a fluorescent protein coding sequence operably linked to the mammalian promoter.

2. The recombinant virus of claim 1, wherein the mammalian promoter is operably linked to a fluorescent sensor molecule coding sequence comprising the fluorescent protein coding sequence.

3. The recombinant virus of claim 2, wherein the fluorescent sensor molecule is an ion-dependent fluorescent sensor.

4. The recombinant virus of claim 2, wherein the fluorescence of the fluorescent sensor molecule is dependent on pH.

5. The recombinant virus of claim 2, wherein the fluorescent sensor molecule is a metal ion-dependent fluorescent sensor.

6. The recombinant virus of claim 5 wherein the metal ion is calcium.

7. The recombinant virus of claim 6, wherein the ion sensor is a cameleon biosensor.

8. The recombinant virus of claim 1, wherein the non-mammalian DNA virus is an invertebrate virus.

9. The recombinant virus of claim 8, wherein the invertebrate virus is a nuclear polyhedrosis virus.

10. The recombinant virus of claim 9, wherein the nuclear polyhedrosis virus is a baculovirus.

11. The recombinant virus of claim 1. wherein a gene required for replication in non-mammalian host cells is deleted or inactivated.

12. The recombinant virus of claim 1, further comprising a coding sequence for a target protein.

13. The recombinant virus of claim 12, wherein the target protein is a G-protein coupled receptor, a kinase, a nuclear receptor, an ion channel, a G-protein, a transporter, a transcription factor, a Glycosidase/glycosyltransferase, a phosphodiesterase, a proteases, or a protein phosphatases.

14. The recombinant virus of claim 1, wherein the mammalian promoter is a liver cell-specific promoter.

15. The recombinant virus of claim 1, wherein the promoter is operably linked to a fusion protein comprising the fluorescent protein and an intracellular targeting sequence.

16. A method for expressing a fluorescent protein, comprising contacting a mammalian cell with a recombinant virus comprising a genome of a non-mammalian DNA virus optionally including genes necessary for the virus to replicate in its natural host, a mammalian promoter, and a fluorescent protein coding sequence operably linked to the mammalian promoter.

17. The method of claim 16, wherein the mammalian promoter is operably linked to a fluorescent sensor molecule coding sequence comprising the fluorescent protein coding sequence.

18. The method of claim 16, wherein the genome of a non-mammalian virus is a baculovirus.

19. The method of claim 16. wherein a gene required for replication in non-mammalian host cells is deleted or inactivated.

20. The method of claim 16, further comprising a coding sequence encoding a G-protein coupled receptor, a kinase, a nuclear receptor, an ion channel, a G-protein, a transporter, a transcription factor, a Glycosidase/glycosyltransferase, a phosphodiesterase, a proteases, or a protein phosphatases.

21. The method of claim 16, wherein the promoter is operably linked to a fusion protein comprising the fluorescent protein and an intracellular targeting sequence.

22. The method of claim 20, further comprising contacting the cells with an on-test molecule.
